(19) United States
(12) Patent Application Publication
(10) Pub. No.: US 2023/0173021 A1
(43) Pub. Date: Jun. 8, 2023

(54) IL-18 Binding Protein (IL-18BP) In Respiratory Diseases

(71) Applicant: AB2 Bio SA, Lausanne (CH)

(72) Inventor: Eduardo Schiffrin, Lausanne (CH)

(21) Appl. No.: 17/998,021

(22) PCT Filed: May 6, 2021
(86) PCT No.: PCT/EP2021/062080

§ 371 Date: Nov. 4, 2022

(30) Foreign Application Priority Data
May 6, 2020 (EP) 20173246.8
Nov. 16, 2020 (EP) 20207684.0

(51) Int. Cl. A61K 38/17; A61P 11/00; A61P 31/14; A61P 29/00; A61K 45/06

(52) U.S. Cl. CPC A61K 38/1709; A61P 11/00; A61P 31/14; A61P 29/00; A61K 45/06

(57) ABSTRACT

The present invention provides means and methods for treating Interleukin 18 (IL-18)-associated respiratory diseases and disorders, particularly means and methods for treating virus-induced IL-18 associated respiratory diseases. In particular, the present invention discloses IL-18 inhibitors such as, for example, the IL-18 binding protein (IL-18BP) for use in the treatment of Interleukin 18 (IL-18)-associated respiratory diseases and disorders such as virus-induced ARDS.

The present invention provides an IL-18 inhibitor for use in the treatment of a respiratory disease, which is caused by virus-induced infection. The present invention discloses IL-18 inhibitors such as, for example, the IL-18 binding protein (IL-18BP) for use in the treatment of a respiratory disease, which is caused by virus-induced infection such as virus-induced ARDS.

Acute respiratory distress syndrome (ARDS) is a life-threatening condition. ARDS is defined by the association of bilateral lung infiltrates and hypoxemia following an initial insult.

ARDS can be caused by different etiologies. The mechanisms of lung injury can be triggered by infections, in the first place, or by immunologic diseases (considered only when infections have been discarded).

Risk factors for the development of ARDS include pneumonia, septicemia, aspiration of gastric content, major trauma, pulmonary contusion, pancreatitis, inhalation injury, severe burns, non-cardiogenic shock, transfusion-associated acute lung injury, pulmonary vasculitis.

Pneumonia is the leading cause of ARDS. The main etiological factors are bacterial and viral, whereas parasites and mycosis are of less importance.

Viruses are responsible for a good percentage (10-30%) of community-acquired pneumonia (CAP). Influenza is a frequent viral etiology of CAP.

Severe CAP in recent epidemics has been associated with respiratory viruses including Rhinovirus, RSV, Parainfluenza, Metapneumovirus, Coronavirus, Enterovirus, Adenovirus Bocavirus, Polyomavirus Herpes simplex virus, and Cytomegalovirus.

In the ARDS diagnostic process the first thing to investigate are the etiological factors responsible for the disease to determine whether it is caused by a respiratory infection, bacterial or viral, an extrapulmonary infection, or a non-infectious process.

Severe and progressive ARDS is characterized by lack of gas exchange improvement, and the presence of systemic manifestations such as systemic inflammatory response syndrome (SIRS), progression of multiple organ dysfunction and high mortality. Several times the systemic manifestations show a Macrophage activation syndrome phenotype.

Elevated levels of pro-inflammatory cytokines are present locally and systemically.

The local inflammatory reaction in ARDS is characterized by high production of cytokines and chemokines that promote leukocyte lung infiltration and inflammatory tissue damage.

Various chemokines such as MCP-1, MIG, IP-10 (CXCL-10) are unusually elevated in infectious ARDS of viral etiology. IL-18 is significantly higher in the more severe cases of ARDS with high mortality rates. The dominant chemokine profile present in these patients is associated with high levels of IL-18 that are, together with IFN-γ, the inducers of the chemokine gene transcription.

Interleukin-18 (IL-18), also known as interferon-gamma inducing factor is a cytokine, which is produced by activated macrophages, Kupffer cells and other cells. IL-18 binds to the IL-18 receptor and induces cell-mediated immunity. Defects (e.g. knock-out) of the IL-18 cytokine receptor or IL-18 cytokine lead to impaired natural killer (NK) cells activity and TH1 responses. Apart from its physiological role, IL-18 may also induce severe inflammatory responses leading to different organ damages including irreversible lung damage.

A new coronavirus named SARS-CoV-2 is responsible for an international outbreak of respiratory illness that ranges from mild self-limited conditions to a severe disease. Three clinical courses of infections can be distinguished: (1) mild illness with upper respiratory tract manifestations, (2) non-life-threatening pneumonia and, (3) severe condition with pneumonia, acute respiratory distress syndrome (ARDS), severe systemic inflammation, organ failures, cardiovascular complications.

The most frequent clinical presentation of the severe form of the disease is characterized by a mild or moderate symptomatology that after about a week progresses to rapid deterioration requiring hospitalization. Patients usually present at this stage with fever, respiratory disease (e.g. cough, dyspnea) and the thorax X-ray shows ground-glass lung infiltrates.

If oxygen saturation is ≤93% on room air or on chronic O2 supplementation, the patient will require supportive care with frequent O2 monitoring.

Upon clinical worsening, recognized by increasing respiratory support (supplementation of O2 requirement), worsening of the X-ray/CT findings, and laboratory markers of systemic inflammation, different treatment options are proposed at different medical centers/hospitals due to the lack of an approved and efficacious recognized treatment.

Some medical centers/hospitals initiated treatment with antiviral agents (remdesivir, chloroquine, hydroxychloroquine, lopinavir, ritonavir (if combined called Kaletra), Interferon-β, interferon-α, ivermectin, etc.). If no control or improvement of the disease is observed after a few days (variable according to different protocols) or disease progression is still ongoing, many medical centers/hospitals will decide to initiate treatment for the control of the inflammatory reaction.

At this stage the clinical condition is characterized by the higher requirement of O2 supply (≥3-liter O2 requirement or any supplement O2 requirement), with appearance of ARDS and/or other organ damage, e.g. renal failure, liver injury, circulatory failure, lung and peripheral thromboembolism, and a further increase of inflammatory markers (high-sensitivity C-reactive protein (hs-CRP)>70-150, ferritin>500 ng/mL).

In the course of the disease an initial virus induced damage seems to be followed by a detrimental inflammatory reaction induced by a cytokine storm. High IL-6 and CRP levels have been reported at this stage of the disease and IL-6 blockade, with different agents, has been the preferred strategy to modulate the inflammatory response. It has been observed though that the response to IL-6 is partial and its efficacy will need confirmation.

Some of the severe patients show another biological profile of inflammation dominated by high levels of ferritin, liver enzymes, thrombocytopenia and other laboratory markers mimicking Macrophage Activation Syndrome (MAS). Moreover, during the SARS-CoV epidemics in 2003, it has been shown that levels of IL-18, IP-10 (CXCL10), MIG, and MCP-1 were very high and were significantly higher in the more severe patients with a high mortality rate (Huang et al., J. of Medical Virology 75:185-194 (2005)).

It was shown within the scope of the present invention that the levels of ferritin and C-reactive protein (CRP) are elevated in SARS-Cov-2 patients, with ferritin levels ranging particularly from 450 to 2500 ng/mL, particularly from 500 to 1500 ng/mL and CRP levies ranging particularly from >5 to 350 μg/mL, particularly from 10 to 150 ng/mL.

It was now shown for the first time within the scope of the present invention that SARS-Cov-2 patients, which show uncontrolled systemic inflammatory reactions, but particular patients that are in need of respiratory assistance, have high levels of total IL-18 and total IL-18BP and, in addition, detectable levels of free IL-18. This shows the strong implication of the IL-18 pathway in patients with severe SARS-Cov-2 infection and supports the use of IL-18 inhibitors in such patients, which block the IL-18 pathway and reduce the IL-18 mediated level of inflammation by binding overshooting amounts of free IL-18 and as a consequence, help avoiding a cytokine release syndrome with the high probability of a fatal outcome.

In particular, IL-18 inhibitors such as, for example, an IL-18 binding protein (IL-18BP) can be used to block the IL-18 activity in virus-induced infectious processes in the lung and thus to interrupt the immunopathological cascade responsible for irreversible lung damage.

The present invention thus provides new opportunities for treating virus-induced IL-18 associated respiratory diseases such as, for example, virus-induce ARDS, by therapeutic targeting and blocking of IL-18 with IL-18 inhibitors such as, for example, an IL-18 binding protein (IL-18BP), which specifically binds free IL-18.

This therapeutic approach may be combined with a precise quantification of free IL-18 in the body fluids and/or body tissues of the patient to be treated by using a quantification assay as decribed in WO 2015/032932 and WO 2016/139297, respectively, the disclosures of which are incorporated herein by reference.

In particular, the present invention provides the following embodiments:
1. An IL-18 inhibitor for use in the treatment of a respiratory disease in a subject, particularly a human, which disease is caused by virus-induced infection.
2. The IL-18 inhibitor for use of embodiment 1, wherein the respiratory disease is Acute Respiratory Distress Syndrome (ARDS).
3. The IL-18 inhibitor for use of embodiment 1 or 2, wherein the virus infection is caused by a virus selected from the group consisting of Rhinovirus, RSV, Influenza virus, Parainfluenza virus, Metapneumovirus, Coronavirus, Enterovirus, Adenovirus, Bocavirus, Polyomavirus, Herpes simplex virus, and Cytomegalovirus.
4. The IL-18 inhibitor for use of embodiment 3, wherein the Coronavirus is of the genus α-CoV, β-CoV, γ-CoV or δ-CoV.
5. The IL-18 inhibitor for use of embodiment 3 or 4, wherein the Coronavirus is selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-CoV or “novel coronavirus 2012”), Severe acute respiratory syndrome coronavirus (SARS-CoV or “SARS-classic”), and Severe acute respiratory syndrome coronavirus 2 (SARS-CoV-2 or “novel coronavirus 2019”).
6. The IL-18 inhibitor for use of embodiment 3, wherein the Influenza virus is of type A, B, C or D, in particular of type A.
7. The IL-18 inhibitor for use of embodiment 3, 4 or 5, wherein the Coronavirus-induced disease is selected from the group consisting of Middle East Respiratory Syndrome (MERS), Severe Acute Respiratory Syndrom (SARS) and COVID-19.
8. The IL-18 inhibitor for use of any one of embodiments 3 to 5 or 7, wherein the Coronavirus is Severe acute respiratory syndrome coronavirus 2 (SARS-CoV-2 or “novel coronavirus 2019”) and the Coronavirus-induced disease is COVID-19.
9. The IL-18 inhibitor for use of any one of embodiments 1-8, wherein treatment is achieved and/or supported by blocking the proinflammatory activity of IL-18.
10. The IL-18 inhibitor for use of any one of embodiments 1 to 9, which is an IL-18 binding protein (IL-18BP), including any functional equivalent or functional part thereof which retains the capability of blocking the proinflammatory activity of IL-18.
11. The IL-18 inhibitor for use of embodiment 10, which is a human IL-18BP (hIL-18 BP), including any functional equivalent or functional part thereof which retains blocking of the proinflammatory activity of IL-18.
12. The IL-18 inhibitor for use of embodiment 11 which is a recombinant human IL-18BP (rhIL-18 BP), including any functional equivalent or functional part thereof which retains the capability of blocking the proinflammatory activity of IL-18.
13. The IL-18 inhibitor for use of embodiment 11 or embodiment 12, wherein said human IL-18BP is selected from isoform a, b, c and d of human IL-18BP, particularly isoform a as in SEQ ID NO: 2, isoform b as in SEQ ID NO: 3, isoform c as in SEQ ID NO: 4 or isoform d as in SEQ ID NO: 5, including any functional equivalent or functional part of isoforms a, b, c and/or d which retains the capability of blocking the proinflammatory activity of IL-18.
14. The IL-18 inhibitor for use of embodiment 11 or 12, which is an IL-18BP as shown in SEQ ID NO: 2, including any functional equivalent or functional part thereof which retains the capability of blocking the proinflammatory activity of IL-18.
15. The IL-18 inhibitor for use of embodiment 14 wherein the functional equivalent has a sequence identity of 80%, 85%, 90%, 91%, 92%, 93%, 94%, 95%, 96%, 97%, 98%, 99% to the sequence depicted in SEQ ID NO: 2 and retains the capability of blocking the proinflammatory activity of IL-18.
16. The IL-18 inhibitor for use of any one of embodiments 10 to 15, wherein the functional equivalent or functional part thereof includes a mutein of IL-18BP, a fragment, a peptide, a functional derivative, a functional fragment, a fraction, a circularly permuted derivative, a fused protein comprising IL-18BP, an isoform or a salt thereof which retains the capability of blocking the proinflammatory activity of IL-18.
17. The IL-18 inhibitor for use of any one of embodiments 10 to 16 comprising in addition to the IL-18 binding protein (IL-18BP), N-terminal and/or C-terminal deletion variants of IL-18BP in an amount of up to 40%, particularly up to 30%, particularly up to 20%, particularly up to 15%, particularly up to 10%, particularly up to 7.5%, particularly up to 5%, particularly up to 2.5%, particularly up to 1%, particularly up to 0.5%, particularly up to 0.25%, particularly up to 0.1%, particularly up to 0.05%, particularly up to 0.01%.
18. The IL-18 inhibitor for use according to embodiment 17, wherein said deletion variants comprise deletions of between 1 and 5 amino acid residues at the C-terminal end of the IL-18BP and/or between 1 and 30 amino acid residues at the N-terminal end of the IL-18BP.
19. The IL-18 inhibitor for use according to embodiment 17 or 18, wherein said N-terminal and/or C-terminal deletion variants of IL-18BP are present in an amount of up to 40%, particularly in an amount of between 2% and 35%.
20. The IL-18 inhibitor for use of any one of embodiments 1 to 19, wherein the body fluids and/or body tissues of the subject to be treated have been quantified to have abnormal levels of free IL-18, which exceed the level of free IL-18 in body fluids and/or body tissues of a healthy control subject, particularly by 5%, 10%, 20%, 30%, 40%, 50%, 60%, 70%, 80%, 90%, 100%, or more than 100%, using an assay capable of detecting free IL-18 in body fluids and/or body tissues, said assay comprising IL-18BP as defined in any one of embodiments 10 to 19 or an antibody or a functional part thereof, which antibody or active part thereof binds to IL-18 at the binding site of IL-18BP or in the vicinity of the binding site of IL-18BP, but does not bind IL-18/IL-18BP complexes.
21. The IL-18 inhibitor for use of embodiment 20, wherein the level of free IL-18 is above the quantification limit, in particular >12 pg/mL, and/or above the detection limit, in particular >4 pg/mL.
22. The IL-18 inhibitor for use of embodiment 20, wherein the level of free IL-18 is two to three times above the level of a healthy control subject.
23. The IL-18 inhibitor for use o any one of embodiments 20 to 22, wherein the assay for quantifying the level of free IL-18 in the body fluids and/or body tissues comprises the following steps:
   a) bringing a sample of body fluid and/or body tissue suspected to contain free IL-18 into contact with the IL-18 inhibitor as defined in any one of embodiments 9 to 19 as the capturing molecule for free IL-18;
   b) allowing the IL-18 inhibitor to bind free IL-18;
   c) detecting the binding of the IL-18 inhibitor and determining the amount of free IL-18 in the sample.
24. The IL-18 inhibitor for use of any one of embodiments 20 to 23, wherein the body fluids and/or body tissues are selected from the group consisting of broncho-alveolar lavage fluid (BALF) circulation fluids, secretion fluids, biopsy, and homogenized tissue, particularly serum, urine, tear, saliva, bile, sweat, exhalation or expiration, sputum, bronchoalveolar fluid, sebum, cellular, gland, mucosa or tissue secretion.
25. A composition for use in the treatment of a respiratory disease as defined in any one of embodiments 1-8 comprising an IL-18 inhibitor as defined in any one of embodiments 9-19 and a pharmaceutically acceptable carrier and/or excipient.
26. The IL-18 inhibitor for use of any one of embodiments 1 to 24 or the composition for use of embodiment 25, wherein said IL-18 inhibitor or composition is administered to a subject in need thereof in a single dose/day, in multiple doses/day, in multiple doses/week or in multiple doses/month.
27. The IL-18 inhibitor for use of any one of embodiments 1 to 24 or the composition for use of embodiment 25, wherein said IL-18 inhibitor or composition is administered in one dose per week, in two doses per week, three doses per week, four doses per week, five doses per week, six doses per week, but particularly seven doses per week, preferably three or four doses per week.
28. The IL-18 inhibitor for use of any one of embodiments 1 to 24 or the composition for use of embodiment 25, wherein said IL-18 inhibitor or composition is administered every 24 h to 48 h, preferably every 48 h.
29. The IL-18 inhibitor for use of any one of embodiments 1 to 24 or the composition for use of embodiment 25, wherein said IL-18 inhibitor or composition is administered in a single dose every other day, for example over 3 weeks.
30. The IL-18 inhibitor for use of any one of embodiments 1 to 24 and 26 to 29 or the composition for use of embodiment 25 to 29, wherein a single dose comprises between 0.5 mg of IL-18 inhibitor/kg body weight and 10 mg IL-18 inhibitor/kg body weight, particularly between 1 mg IL-18 inhibitor/kg body weight and 8 mg IL-18 inhibitor/kg body weight, particularly between 1,5 mg IL-18 inhibitor/kg body weight and 6 mg IL-18 inhibitor/kg body weight, particularly between 2 mg IL-18 inhibitor/kg body weight and 4 mg IL-18 inhibitor/kg body weight.
31. The IL-18 inhibitor for use of any one of embodiments 1 to 24 and 26 to 29 or the composition for use of embodiment 24-28, wherein a single dose of between 0.5 mg IL-18 inhibitor/kg body weight and 5 mg IL-18 inhibitor/kg body weight is administered every 24 or 48 h, particularly, wherein a single dose of 2 mg IL-18 inhibitor/kg body weight is administered every 48 h.
32. The IL-18 inhibitor for use of any one embodiments 9 to 19, which is a recombinant human IL-18BP (rhlL-18 BP), including any functional equivalent or functional part thereof or the composition for use of embodiment 25 comprising a recombinant human IL-18BP (rhlL-18 BP), including any functional equivalent or functional part thereof, which retains the capability of blocking the proinflammatory activity of free IL-18, wherein the recombinant human IL-18BP (rhlL-18 BP) or the composition comprising the recombinant human IL-18BP (rhlL-18 BP) is administered in a single dose every other day of 2 mg/kg body weight.
33. The IL-18 inhibitor for use of any one of embodiments 1-24 and 26-32 or the composition for use of embodiment 25-32, for the treatment of a subject, particularly a human, with confirmed SARS-CoV-2 infection undergoing systemic inflammatory reaction and in need of respiratory assistance.
34. The IL-18 inhibitor for use of any one of embodiments 1 to 24 and 26 to 33 or the composition for use of any one of embodiments 24 to 33, for the treatment of a patient suffering from SARS-Cov-2 showing uncontrolled systemic inflammatory reactions and/or high levels of total IL-18 and/or total IL-18BP and/or detectable levels of free IL-18.
35. The IL-18 inhibitor for use of any one of embodiments 1 to 24 and 26 to 34 or the composition for use of any one of embodiments 25 to 34, wherein the IL-18 inhibitor and/or the composition are used in co-medication, preferably with an anti-viral agent and/or an anti-inflammatory agent and/or an immunosuppressant and/or a monoclonal antibody and/or a cocktail of monoclonal antibodies and/or a vasopressor and/or an anticoagulant and/or a vasodilator and/or a mucolytic.
36. The IL-18 inhibitor for use or the composition for use of embodiment 35, wherein the anti-viral agent is selected from the group consisting of Remdesivir, Chloroquine, Hydroxychloroquine, Lopinavir, Ritonavir, Interferon-β, Interferon-α, and Ivermectin.
37. The IL-18 inhibitor for use or the composition for use of embodiment 35, wherein the anti-inflammatory agent and the immunosuppressant is selected from the group consisting of Corticosteroids, intravenous immunoglobulin, Tocilizumab (anti-IL-6 receptor), sarilumab (anti-IL-6 receptor), canakinumab (anti-IL-1 beta) anakinra (recombinant IL-1ra), Emapalumab (anti-IFNγ), JAK inhibitors.
38. The IL-18 inhibitor for use or the composition for use of embodiment 35, wherein the monoclonal antibody is selected from REGN-COV2, LY-COV55, and CT-P59.
39. The IL-18 inhibitor for use of any one of embodiments 1 to 24 and 26 to 38 or the composition for use of any one of embodiments 25 to 38, wherein the subject to be treated is a mammal.
40. The IL-18 inhibitor for use or the composition for use of embodiment 39, wherein the subject to be treated is a human.
41. A recombinant human IL-18BP (rhIL-18 BP) or a composition comprising the recombinant human IL-18BP (rhIL-18 BP), including any functional equivalent or functional part thereof which retains the capability of blocking the proinflammatory activity of IL-18, for use in the treatment of COVID-19 in a human, optionally a human with confirmed SARS-CoV-2 infection and, optionally, wherein the human has a detectable level of free IL-18, particularly wherein said recombinant human IL-18BP (rhIL-18 BP) or the composition comprising the recombinant human IL-18BP (rhIL-18 BP) is administered to said patient in a single dose every other day of 2 mg/kg body weight.
42. The recombinant human IL-18BP (rhIL-18 BP) or a composition comprising the recombinant human IL-18BP (rhlL-18 BP), including any functional equivalent or functional part thereof which retains the capability of blocking the proinflammatory activity of IL-18, for use according to embodiment 41, wherein said recombinant human IL-18BP (rhIL-18 BP) or the composition comprising the recombinant human IL-18BP (rhlL-18 BP) is administered to said human patient in a single dose every other day of 2 mg/kg body weight.
43. The recombinant human IL-18BP or the composition comprising the recombinant human IL-18BP (rhIL-18 BP), including any functional equivalent or functional part thereof for use according to embodiment 41 or embodiment 42, wherein the human patient suffering from SARS-Cov-2 shows uncontrolled systemic inflammatory reactions and has high levels of total IL-18 and total IL-18BP and, in addition, detectable levels of free IL-18.
44. The recombinant human IL-18BP or the composition comprising the recombinant human IL-18BP (rhlL-18 BP), including any functional equivalent or functional part thereof for use according to embodiment 43, wherein the human patient is in need of respiratory assistance and/or the level of free IL-18 is above the quantification limit (>12 pg/mL) and/or above the detection limit (>4 pg/mL).
45. The recombinant human IL-18BP or the composition comprising the recombinant human IL-18BP (rhlL-18 BP), including -any functional equivalent or functional part thereof for use according to embodiment 41 or 42, wherein the level of free IL-18 in the body fluids or body tissues of the human patient is two to three times above the level of a healthy control subject.

The present invention relates to the IL-18 inhibitor as defined above in the various embodiments or to the composition comprising the IL-18 inhibitor, particularly for use according to the various embodiments of the invention as described herein.

Particularly, the IL-18 inhibitor is an inhibitor, which blocks the proinflammatory activity of IL-18 and/or has capablility of blocking the proinflammatory activity of IL-18.

In a specific embodiment, said IL-18 inhibitor is an IL-18 binding protein (IL-18BP), particularly human IL-18BP (hIL-18BP), particularly recombinant human IL-18BP (rhIL-18BP), including any functional equivalent or functional part thereof, which retains the capability of blocking the proinflammatory activity of IL-18.

In another specific embodiment, the IL-18BP is selected from isoform a, b, c and d of IL-18BP, particularly isoform a, particularly isoform c, particularly isoform a, b, c or d as shown in SEQ ID NOs 2, and SEQ ID NOs: 3, 4 and 5, but especially isoform a of IL-18BP as shown in SEQ ID NO: 2, or isoform c as shown in SEQ ID NO: 4, including any functional equivalent or functional part thereof, which retains the capability of blocking the proinflammatory activity of IL-18.

Also mixtures of the above isoforms in different combinations may be used in the composition of the invention, but particularly a mixture of isoform a and isoform c, including any functional equivalent or functional part thereof which retains the capability of blocking the proinflammatory activity of IL-18.

Within the present invention, the term “functional” is meant to relate to the equivalent or the part that has still retained the IL-18 blocking activity of the IL-18 inhibitor in virus-induced infectious processes in the lung and is thus capable of blocking the proinflammatory activity of IL-18 and thus to interrupt the immunopathological cascade responsible for irreversible lung damage.

Also comprised within the scope of the present invention is a functional mutein of IL-18BP, a functional fragment, a functional peptide, a functional derivative, a functional fraction, a functional circularly permuted derivative, a functional fused protein comprising IL-18BP, an functional isoform or a functional salt thereof, which retains the capability of blocking the proinflammatory activity of IL-18, preferably which retains the IL-18 blocking activity of the IL-18BP in virus-induced infectious processes in the lung and is thus capable of blocking the proinflammatory activity of free IL-18 and thus to interrupt the immunopathological cascade responsible for irreversible lung damage.

In one embodiment, the invention relates to an IL-18BP, which is a fused protein comprising all or part of an IL-18BP, fused to all or part of an immunoglobulin, preferably to the constant region of an immunoglobulin, and wherein the fused protein is still capable of binding to IL-18, which retains the capability of blocking the proinflammatory activity of free IL-18, and preferably has retained the IL-18 blocking activity of the IL-18BP in virus-induced infectious processes in the lung and is thus capable of blocking the proinflammatory activity of free IL-18 and thus to interrupt the immunopathological cascade responsible for irreversible lung damage. More specifically, the immunoglobulin may be of the IgG1 or IgG2 isotype, for use in a composition according to any one of the embodiments described herein.

In another embodiment the invention relates to an IL-18BP, which is a fused protein comprising all or part of an IL-18BP, fused to all or part of a small molecule, particularly to all or part of a small molecular drug, wherein the fused protein retains the capability of blocking the proinflammatory activity of free IL-18, and preferably is still capable of blocking activity of the IL-18BP in virus-induced infectious processes in the lung and is thus capable of blocking the proinflammatory activity of IL-18 and thus to interrupt the immunopathological cascade responsible for irreversible lung damage.

In one embodiment, the present invention provides the IL-18 inhibitor for use as disclosed in any one of the embodiments described herein, wherein the inhibitor is an IL-18 Binding Protein (IL-186P) which has a sequence identity of 80%, 81%, 82%, 83%, 84%, 85%, 86%, 87%, 88%, 89%, 90%, 91%, 92%, 93%, 94%, 95%, 96%, 97%, 98%, 99% or 100% to the sequence depicted in SEQ ID NO: 2 and SEQ ID NOs: 3, 4 and 5, but particularly to the sequence depicted in SEQ ID NO: 2 and SEQ ID NO: 4 and has retained the IL-18 blocking activity of the IL-18 inhibitor in virus-induced infectious processes in the lung and is thus capable of blocking the proinflammatory activity of IL-18 and thus to interrupt the immunopathological cascade responsible for irreversible lung damage.

In another embodiment of the invention, the IL-18BP or the composition comprising the IL-18BP may in addition comprise N-terminal and/or C-terminal deletion variants of IL-18BP in an amount of up to 40%, particularly up to 30%, particularly up to 20%, particularly up to 15%, particularly up to 10%, particularly up to 7.5%, particularly up to 5%, particularly up to 2.5%, particularly up to 1%, particularly up to 0.5%, particularly up to 0.25%, particularly up to 0.1%, particularly up to 0.05%, particularly up to 0.01%.

In a specific embodiment, said N-terminal and/or C-terminal deletion variants of IL-18BP are present in an amount of up to 40%, particularly in an amount of between 2% and 35%.

In particular, said deletion variants comprise deletions of between 1 and 5 amino acid residues at the C-terminal end of the IL-18BP and/or between 1 and 30 amino acid residues at the N-terminal end of the IL-18BP.

In one embodiment, the invention relates to a composition for use in the treatment of a respiratory disease as defined in any one of the embodiments disclosed herein, particularly embodiments 1-8, comprising an IL-18 inhibitor as defined in any one of the embodiments disclosed herein, particularly embodiments 9-19, and a pharmaceutically acceptable carrier and/or excipient.

In various embodiments of the invention, the IL-18 inhibitor, particularly the IL-18BP of the invention as described herein, including any functional equivalent or functional part thereof, or the composition comprising said IL-18 inhibitor, particularly the IL-18BP of the invention, including any functional equivalent or functional part thereof, is used for the treatment of a virus-induced respiratory disease, particularly of a virus-induced Acute Respiratory Distress Syndrome (ARDS).

The inducing virus may be an RNA virus or a DNA virus.

In one embodiment of the invention, the IL-18 inhibitor, particularly the IL-18BP of the invention, including any functional equivalent or functional part thereof, or the composition comprising said IL-18 inhibitor, particularly the IL-18BP of the invention, including any functional equivalent or functional part thereof, is used for the treatment of a DNA virus-induced respiratory disease, particularly a virus-induced ARDS.

In a specific embodiment, the virus-induced respiratory disease, particularly the virus-induced ARDS, is caused by a DNA virus selected from the group consisting of Adenovirus, Rhinovirus, RSV, Influenza virus, Parainfluenza virus, Metapneumovirus, Coronavirus, Enterovirus, Adenovirus, Bocavirus, Polyomavirus, Herpes simplex virus, and Cytomegalovirus, Bocavirus, Polyomavirus, Herpes simplex virus, and Cytomegalovirus.

In another specific embodiment, the IL-18 inhibitor, particularly the IL-18BP of the invention, including any functional equivalent or functional part thereof, or the composition comprising said IL-18 inhibitor, particularly the IL-18BP of the invention, including any functional equivalent or functional part thereof, is used for the treatment of a RNA virus-induced respiratory disease, particularly a RNA virus-induced ARDS.

The RNA virus may be an enveloped or coated virus or a nonenvaloped or naked RNA virus.

In particular, the RNA virus may be single stranded RNA (ssRNA) virus or a double stranded RNA (dsRNA) virus.

The single stranded RNA virus may be a positive sense ssRNA virus or a negative sense ssRNA virus.

In a specific embodiment, the virus-induced respiratory disease, particularly the virus-induced ARDS, is caused by a RNA virus selected from the group consisting of Rhinovirus, RSV, Influenza virus, Parainfluenza virus, Metapneumovirus, Coronavirus, Enterovirus Adenovirus, Bocavirus, Polyomavirus, Herpes simplex virus, and Cytomegalovirus.

In a specific embodiment, the virus-induced respiratory disease, particularly the virus-induced ARDS, is caused by a Coronavirus, particularly a Cornavirus of the genus α-CoV, β-CoV, γ-CoV or δ-CoV.

In another specific embodiment, the Cornavirus is of the genus α-CoV or β-CoV.

In particular, the Coronavirus is selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-CoV or “novel coronavirus 2012”), Severe acute respiratory syndrome coronavirus (SARS-CoV or “SARS-classic”), Severe acute respiratory syndrome coronavirus 2 (SARS-CoV-2 or “novel coronavirus 2019”), Variant of Concern (SARS-CoV-2 VOC 202012/01), Severe acute respiratory syndrome coronavirus 2 variant 501Y.V2 (SARS-CoV-2 501Y.V2 or SARS-CoV-2 B 1.351), B.1.1.7 variant, B.1.427 variant, and Severe acute respiratory syndrome coronavirus 2 P1 (SARS-CoV-2 P1).

In a specific embodiment, the IL-18 inhibitor, particularly the IL-18BP of the invention, including any functional equivalent or functional part thereof, or the composition comprising said IL-18 inhibitor, particularly the IL-18BP of the invention, including any functional equivalent or functional part thereof, is used for the treatment of a Coronavirus-induced respiratory disease selected from the group consisting of Middle East Respiratory Syndrome (MERS), Severe Acute Respiratory Syndrom (SARS) and COVID-19.

In another specific embodiment, the IL-18 inhibitor, particularly the IL-18BP of the invention, including any functional equivalent or functional part thereof, or the composition comprising said IL-18 inhibitor, particularly the IL-18BP of the invention, including any functional equivalent or functional part thereof, is used for the treatment of a Coronavirus-induced respiratory disease, wherein the Coronavirus is Severe acute respiratory syndrome coronavirus 2 (SARS-CoV-2 or “novel coronavirus 2019”) and the Coronavirus-induced disease is Covid-19.

In another specific embodiment, the virus-induced respiratory disease, particularly the virus-induced ARDS, is caused by an Influenza virus, particularly an Influenza virus of type A, B, C or D, particularly Influenza virus type A.

The IL-18 inhibitor, particularly the IL-18BP of the invention, including any functional equivalent or functional part thereof, or the composition comprising said IL-18 inhibitor, particularly the IL-18BP of the invention, including any functional equivalent or functional part thereof, is used for the treatment of one or more of the above identified respiratory diseases in a mammal, particularly in a human.

Treatment of any of the IL-18 associated disease or disorder according to any one of the preceding embodiments with the IL-18 inhibitor of the invention, particularly the IL-18BP of the invention as defined herein, including any functional equivalent or functional part thereof, or the composition comprising the IL-18 inhibitor of the invention, particularly the IL-18BP of the invention as defined herein, including any functional equivalent or functional part thereof, comprises prevention, halting, alleviation or reversion of symptoms associated with said disease or disorder.

In another specific embodiment, the IL-18 inhibitor, particularly the IL-18BP, of the invention, including any functional equivalent or functional part thereof, or the composition comprising the IL-18 inhibitor, particularly the IL-18BP, of the invention, including any functional equivalent or functional part thereof, is administered to the subject to be treated at least until the treated subject shows a therapeutic response.

In various further embodiment, the invention relates to the IL-18 inhibitor of the invention, particularly the IL-18BP of the invention as defined herein, or the composition comprising the IL-18 inhibitor of the invention, particularly the IL-18BP of the invention as defined herein, for use according to any one of the preceding embodiments, wherein
increased expression of IFNγ, IL-13 or IL-17A is modified, particularly inhibited, compared to untreated subjects suffering from said disease or disorder; and/or
binding of free IL-18 by the IL-18BP compensates the IL-18/IL-18BP imbalance by trapping and neutralizing the excess of free IL-18 in tissue and circulation; and/or
the IL-18BP inhibits infiltration of neutrophils into the lung, particularly through mitigation of G-CSF release in the lung airways; and/or
IL-18 binding is restricted or inhibited, particularly binding of free IL-18 to IL-18 receptor (IL-18R), but especially free IL-18 binding to IL-18Rα; and/or
the IL-18BP reduces binding of IL-18 to IL-18 receptor, particularly binding to IL-18Rα by at least 5%, particularly by at least 10%, particularly by at least 15%, particularly by at least 20%, particularly by at least 25%, particularly by at least 30%, particularly by at least 40%, particularly by at least 45%, particularly by at least 50%, particularly by at least 55%, particularly by at least 60%, particularly by at least 65%, particularly by at least 70, particularly by at least 75, particularly by at least 80, particularly by at least 85%, particularly by at least 90%, particularly by at least 95%, particularly by 100%; and/or
the IL-18BP neutralizes free IL-18 by restricting or preventing IL-18 binding to IL-18 receptor (IL-18R), especially free IL-18 binding to IL-18Rα.

In one embodiment, the IL-18 inhibitor is provided for use of any one of the embodiments provided herein, wherein the subject has been diagnosed with a level of ferritin (FERR) above 400 ng/mL, preferably above 1000 ng/mL, above 1500 ng/mL, above 2000 ng/mL, above 2500 ng/mL, above 3000 ng/mL, or above 3500 ng/mL.

In one embodiment, the invention relates to the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, for use according to any one of the preceding embodiments, wherein the level of free IL-18 in the body fluids and/or body tissues has been determined to be ≥5 pg/mL and, particularly, up to 10000 pg/mL as compared to ≤4 pg/mL in the healthy control.

In a specific embodiment of the invention, the level of free IL-18 in the body fluids and/or body tissues is above the quantification limit (>12 pg/mL).

In another specific embodiment of the invention, the level of free IL-18 in the body fluids and/or body tissues is above the detection limit (>4 pg/mL).

In still another specific embodiment, the level of free IL-18 in the body fluids and/or body tissues is two to three times above normal.

The determination of free IL-18 in the body fluids and/or body tissues may be accomplished by using an assay for quantifying the level of free IL-18 in the body fluids and/or body tissues in a body sample or in situ, which includes the steps of:

a) bringing a sample of body fluid and/or body tissue or a body part or body area suspected to contain free IL-18 into contact with IL-18BP or an antibody, which specifically binds to free IL-18, but not to IL-18 bound in a complex and functions as the capturing molecule for free IL-18;

b) allowing the IL-18BP or the antibody to bind to free IL-18;

c) detecting the binding of IL-18 to the IL-18BP or the antibody and determining the amount of free IL-18 in the sample or in situ.

In one embodiment, the invention relates to the method according to any one of the preceding embodiments, wherein said sample is selected from the group consisting of broncho-alveolar lavage fluid (BALF) circulation fluids, secretion fluids, biopsy, and homogenized tissue, particularly serum, urine, tear, saliva, bile, sweat, exhalation or expiration, sputum, bronchoalveolar fluid, sebum, cellular, gland, mucosa or tissue secretion.

For determining the presence or absence of free IL-18 in a sample according to the method described herein in the various embodiments, any immunoassay format known to those of ordinary skill in the art. may be used such as, for example, assay formats which utilize indirect detection methods using secondary reagents for detection, In particular, ELISA's and immunoprecipitation and agglutination assays may be used. A detailed description of these assays is, for example, given in Harlow and Lane, Antibodies: A Laboratory Manual (Cold Spring Harbor Laboratory, New York 1988 555-612, WO96/13590 to Maertens and Stuyver, Zrein et al. (1998) and WO96/29605.

The sample may be a non-diluted or diluted biological fluid, such as, without being restricted thereto, serum, urine, tear, saliva, bile, sweat, exhalation or expiration, sputum, bronchoalveolar fluid, sebum, cellular, gland, mucosa or tissue secretion, biopsy, homogenized tissue.

For in situ diagnosis, the IL-18BP or the antibody or any active and functional part thereof may be administered to the organism to be diagnosed by methods known in the art such as, for example, intravenous, intranasal, intraperitoneal, intracerebral, intraarterial injection such that a specific binding between the IL-18BP or the antibody with free IL-18 may occur. The antibody/antigen complex may conveniently be detected through a label attached to the antibody or a functional fragment thereof or any other art-known method of detection.

In another aspect of the invention, detection of free IL-18 described herein may be accomplished by an immunoassay procedure. The immunoassay typically includes contacting a test sample with an antibody or the IL-18BP as described herein in the various embodiments that specifically binds to free IL-18, and detecting the presence of the the IL-18BP/free IL-18 complex or the antibody/free IL-18 complex in the sample. The immunoassay procedure may be selected from a wide variety of immunoassay procedures known to those skilled in the art such as, for example, competitive or non-competitive enzyme-based immunoassays, enzyme-linked immunosorbent assays (ELISA), radioimmunoassay (RIA), and Western blots, etc. Further, multiplex assays may be used, including arrays, wherein IL-18BP or the antibody are placed on a support, such as a glass bead or plate, and reacted or otherwise contacted with the test sample.

Antibodies used in these assays may be monoclonal or polyclonal, and may be of any type such as IgG, IgM, IgA, IgD and IgE. Antibodies may be produced by immunizing animals such as rats, mice, and rabbits. The antigen used for immunization may be isolated from the samples or synthesized by recombinant protein technology. Methods of producing antibodies and of performing antibody-based assays are well-known to the skilled artisan and are described, for example, more thoroughly in Antibodies: A Laboratory Manual (1988) by Harlow & Lane; Immunoassays: A Practical Approach, Oxford University Press, Gosling, J. P. (ed.) (2001) and/or Current Protocols in Molecular Biology (Ausubel et al.) which is regularly and periodically updated.

Various chemical or biochemical derivatives of the IL-18BP or antibodies or antibody fragments can be produced using known methods. One type of derivative which is diagnostically useful as an immunoconjugate comprising an IL-18BP or an antibody molecule, or an antigen-binding fragment thereof, to which is conjugated a detectable label. However, in many embodiments, the IL-18BP or the antibody is not labeled but in the course of an assay, it becomes indirectly labeled by binding to or being bound by another molecule that is labeled. The invention encompasses molecular complexes comprising an IL-18BP or an antibody molecule and a label.

Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferones, fluoresceins, fluorescein isothiocyanate, rhodamines, dichlorotriazinylamine fluorescein, dansyl chloride, phycoerythrins, Alexa Fluor 647, Alexa Fluor 680, DilC₁₉(3), Rhodamine Red-X, Alexa Fluor 660, Alexa Fluor 546, Texas Red, YOYO-1+DNA, tetramethylrhodamine, Alexa Fluor 594, BODIPY FL, Alexa Fluor 488, Fluorescein, BODIPY TR, BODIPY TMR, carboxy SNARF-1, FM 1-43, Fura-2, Indo-1, Cascade Blue, NBD, DAPI, Alexa Fluor 350, aminomethylcoumarin, Lucifer yellow, Propidium iodide, or dansylamide; an example of a luminescent material includes luminol; examples of bioluminescent materials include green fluorescent proteins, modified green fluorescent proteins, luciferase, luciferin, and aequorin, and examples of suitable radioactive material include ¹²⁵I, ¹³¹I, ³⁵S or ³H.

The immunoassays will typically comprise incubating a sample, such as a biological fluid, a tissue extract, freshly harvested cells, or lysates of cells, in the presence of a detectably labeled IL-18BP or an antibody or peptide fragments thereof, and detecting the bound IL-18BP or antibody by any of a number of techniques well-known in the art. One way of measuring the level of free IL-18 with the IL-18BP or antibody antibody is by enzyme immunoassay (EIA) such as an enzyme-linked immunosorbent assay (ELISA) (Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., 1980). The enzyme, either conjugated to the IL-18BP or the antibody or to a binding partner for the IL-18BP or the antibody, when later exposed to an appropriate substrate, will react with the substrate in such a manner as to produce a chemical moiety which can be detected, for example, by spectrophotometric, or fluorimetric means.

In a specific embodiment of the invention, the IL-18BP used in any of the above formats, but particularly in an ELISA format is IL-18BP isoform a, b, c or d, or a functional equivalent or a functional derivate thereof, or a functional fragment thereof, particularly isoform a, particularly isoform c, or a derivate thereof, particularly isoform a, b, c or d as shown in SEQ ID NOs 2, and 3, 4 and 5, but especially the isoform a of IL-18BP as shown in SEQ ID NO: 2 or the isoform c as shown in SEQ ID NO 4.

Also mixtures of the above isoforms may be used in the composition of the invention, but particularly a mixture of isoform a and isoform c.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled IL-18BP or antibody. The solid phase support may then be washed with the buffer a second time to remove unbound IL-18BP or antibody. The amount of bound label on solid support may then be detected by conventional means. A well-known example of such a technique is Western blotting.

In various embodiments, the present invention provides compositions comprising labeled IL-18BP or labelled antibodies according to the invention as described herein.

In still another embodiment, the invention relates to a method for treating a respiratory disease as defined in any one of the preceding embodiments, said method comprising:
a. in a first optional step, quantifying the amount of free IL-18 in the body fluids and/or body tissues of said subject using the method according to the invention and as described herein in the various embodiments;
b. administering to a subject, which suffers from a respiratory disease as defined in any one of the preceding embodiments and has abnormal levels of free IL-18 in the body fluids and/or body tissues, which exceed the level of free IL-18 in body fluids and/or body tissues of a healthy control subject by 5%, 10%, 20%, 30%, 40%, 50%, 60%, 70%, 80%, 90%, 100%, or more than 100%, a therapeutically or prophylactically effective amount of an IL-18 inhibitor or a composition comprising said IL-18 inhibitor as defined in any one of the preceeding embodiments, particularly by systemic, intranasal, buccal, oral, transmucosal, intratracheal, intravenous, subcutaneous, intraurinary tract, intravaginal, sublingual, intrabronchial, intrapulmonary, transdermal or intramuscular administration, in particular broncho-pulmonary administration.

In a specific embodiment, the IL-18 inhibitor is the IL-18BP as defined herein in the preceeding embodiments.

The IL-18 inhibitor or the pharmaceutical composition comprising the the IL-18 inhibitor of the invention and as disclosed herein in the various embodiments is administered to a subject suffering from a respiratory disease as defined herein before in any of the preceeding embodiments in suitable dosage forms and units and dosage intervals.

In a specific embodiment, the IL-18 inhibitor, particularly the IL-18BP, of the invention, including any functional equivalent or functional part thereof, as defined herein is formulated as a pharmaceutical composition comprising a sterile solution for injection and further sodium chloride, and/or sodium hydroxide and/or sodium phosphate buffer, particularly in a concentration of between 0.01 M and 0.1 M, particularly between 0.01 M and 0.05 M, but especially of 0.01 M.

In particular, said composition of the invention comprises sodium chloride, sodium hydroxide and a sodium phosphate buffer in a concentration of 0.01 M.

The IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, may be administered to a patient in need thereof in a single dose or dosage unit/day, in multiple doses or doseage units/day, in multiple doses or doseage units/week or in multiple doses or doseage units/month. The single dose or dosage unit may be split into several doses or dosage units and administered to the subject to be treated over several hours or a whole day.

In one embodiment, the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, is administered in one dose per week, in two doses per week, three doses per week, four doses per week, five doses per week, six doses per week, but particularly seven doses per week.

In another embodiment, the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, is administered every 24 h to 48 h.

In still another embodiment, the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, is administered in every other day, three or four times per week.

In one embodiment, the composition according to the invention will be administered by s.c. injection. In particular, the site of the s.c. injection is alternated, particularly the site of injection is outside of the thighs and the various quadrants of the anterior abdominal wall. The separate injections that constitute a single dosage of the composition of the invention is particularly administered within the same body region but not at the exact same injection site.

In one embodiment, the composition is brought to room temperature, particularly between 18-25° C., before administration.

In a specific embodiment, a single dose of the composition of the invention and particularly the composition for use according to any one of the preceding embodiments comprises between 10 mg and 600 mg IL-18BP.

In particular, the single dose comprises between 10 and 20 mg, between 20 and 40 mg, between 40 and 80 mg, between 80 and 160 mg, between 160 mg and 320 mg or between 320 mg and 600 mg IL-18BP.

In various embodiments of the invention, a single dose comprises between 0.5 mg of IL-18 inhibitor/kg body weight and 10 mg IL-18 inhibitor/kg body weight, particularly between 1 mg IL-18 inhibitor/kg body weight and 8 mg IL-18 inhibitor/kg body weight, particularly between 1.5 mg IL-18 inhibitor/kg body weight and 6 mg IL-18 inhibitor/kg body weight, particularly between 2 mg IL-18 inhibitor/kg body weight and 4 mg IL-18 inhibitor/kg body weight.

In a specific embodiment of the invention, a single dose comprises between 2 mg of IL-18BP/kg body weight and 3 mg IL-18BP/kg body weight, particularly 2 mg of IL-18BP/kg body weight.

In a specific embodiment, the IL-18 inhibitor, particularly the IL-18BP, of the invention, including any functional equivalent or functional part thereof, or the composition comprising the IL-18 inhibitor, particularly the IL-18BP, of the invention, including any functional equivalent or functional part thereof is administered in a dosage of between 0.5 mg IL-18 inhibitor/kg body weight and 5 mg IL-18 inhibitor/kg body weight every 24 or 48 h, particularly, a single dose of 2 mg IL-18 inhibitor/kg body weight is administered every 48 h.

In another specific embodiment, human IL-18BP (rhlL-18 BP), including any functional equivalent or functional part thereof or the composition comprising a recombinant human IL-18BP (rhlL-18 BP), including any functional equivalent or functional part thereof, which retains the capability of blocking the proinflammatory activity of IL-18 is used in the treatment of a virus-induced respiratory disease as described herein in the various embodiments, wherein the recombinant human IL-18BP (rhIL-18 BP) or the composition comprising the recombinant human IL-18BP (rhIL-18 BP) is administered in a single dose every other day of 2 mg/kg body weight, for example over three weeks.

In still another specific embodiment, a recombinant human IL-18BP (rhlL-18 BP) or a composition comprising the recombinant human IL-18BP (rhIL-18 BP), including any functional equivalent or functional part thereof which retains the capability of blocking the proinflammatory activity of IL-18, is used in the treatment of COVID-19 in a human patient, which suffers from COVID-19 and has a level of free IL-18 two to three times above the level of a healthy control subject, wherein said recombinant human IL-18BP (rhIL-18 BP) or the composition comprising the recombinant human IL-18BP (rhIL-18 BP) is administered to said patient in a single dose every other day, three or four times per week, of 2 mg/kg body weight, for example over three weeks.

The compositions of the invention may comprise additional medicinal agents, pharmaceutical agents, carriers, buffers, dispersing agents, diluents, co-therapeutic agents such as anti-inflammatory, bronchodilatory, antihistamine, decongestant, anti-tussive drug substances, antiviral and/or immunosuppressant drugs, and the like, depending on the intended use and application.

In one embodiment of the present invention, the IL-18 inhibitor or the pharmaceutical composition comprising the the IL-18 inhibitor of the invention and as disclosed herein in the various embodiments, is administered prophylactically.

In another embodiment of the present invention, the IL-18 inhibitor or the pharmaceutical composition comprising the the IL-18 inhibitor of the invention and as disclosed herein in the various embodiments is administered therapeutically.

In one embodiment, the IL-18 inhibitor, particularly the IL-18BP, of the invention, including any functional equivalent or functional part thereof, or the composition comprising the IL-18 inhibitor, particularly the IL-18BP, of the invention, including any functional equivalent or functional part thereof is used in co-medication. Preferably co-medication may be with an anti-viral agent and/or an anti-inflammatory agent and/or an immunosuppressant and/or a monoclonal antibody and/or a cocktail of monoclonal antibodies and/or a vasopressor and/or an anticoagulant and/or a vasodilator and/or a mucolytic.

The anti-viral agent may be selected from the group consisting of Remdesivir, Chloroquine, Hydroxychloroquine, Lopinavir, Ritonavir, Interferon-β, Interferon-α, and Ivermectin.

The anti-inflammatory agent and the immunosuppressant may be selected from the group consisting of Corticosteroids, intravenous immunoglobulin, Tocilizumab (anti-IL-6 receptor), anakinra (recombinant IL-1ra), JAK inhibitors.

The monoclonal antibody may be selected from REGN-COV2, LY-COV555 (bamlanivimab), and CTp-59.

In one embodiment of the present invention, the IL-18 inhibitor or the pharmaceutical composition comprising the the IL-18 inhibitor of the invention and as disclosed herein in the various embodiments is administered to a subject suffering from a respiratory disease as defined herein before in any of the preceeding embodiments by systemic, intranasal, intraocular, intravitral, eye drops, buccal, oral, transmucosal, intratracheal, intravenous, subcutaneous, intraurinary tract, intrarectal, intravaginal, sublingual, intrabronchial, intrapulmonary, transdermal or intramuscular administration. In particular, the administration is done by subcutanous injection or broncho-pulmonary application.

The IL-18 inhibitor or the pharmaceutical composition comprising the IL-18 inhibitor of the invention and as disclosed herein in the various embodiments may be provided as a liquid, liquid spray, microspheres, semisolid, gel, or powder for transmucosal administration, e.g. intranasal, buccal, oral transmucosal, intratracheal, intraurinary tract, intravaginal, sublingual, intrabronchial, intrapulmonary and/or transdermal administration. Further, the composition may be in a solid dosage form for buccal, oral transmucosal and/or sublingual administration. Intranasal, buccal, oral intratracheal, intraurinary tract, intravaginal, transmucosal and sublingual administrations lead to the disintegration of the composition as described herein in an oral cavity at body temperature and optionally may adhere to the body tissue of the oral cavity. Additionally, the composition as disclosed herein further may include one or more excipient, diluent, binder, lubricant, glidant, disintegrant, desensitizing agent, emulsifier, mucosal adhesive, solubilizer, suspension agent, viscosity modifier, ionic tonicity agent, buffer, carrier, surfactant, flavor, or mixture thereof.

In a specific aspect of the present invention, the IL-18 inhibitor or the pharmaceutical composition comprising the IL-18 inhibitor is formulated as a parenteral, or intravenous solution, suspension, emulsion, as a tablet, pill, bioadhesive patch, drops, sponge, film, lozenge, hard candy, wafer, sphere, lollipop, disc-shaped structure, suppository or spray.

Transmucosal administration is generally rapid because of the rich vascular supply to the mucosa and the lack of a stratum corneum in the epidermis. Such drug transport typically provides a rapid rise in blood concentrations, and similarly avoids the enterohepatic circulation and immediate destruction by gastric acid or partial first-pass effects of gut wall and hepatic metabolism. Drugs typically need to have prolonged exposure to a mucosal surface for significant drug absorption to occur.

The transmucosal routes can also be more effective than the oral route in that these routes can provide for relatively faster absorption and onset of therapeutic action. Further, the transmucosal routes can be preferred for use in treating patients who have difficulty in swallowing tablets, capsules, or other oral solids, or those who have disease-compromised intestinal absorption. Accordingly, there are many advantages to transmucosal administration of the IL-18 inhibitor, particularly the IL-18BP, or a pharmaceutical composition comprising the IL-18 inhibitor, particularly the IL-18BP and a pharmaceutically acceptable carrier and/or excipient.

In either of the intranasal or buccal routes, drug absorption can be delayed or prolonged, or uptake may be almost as rapid as if an intravenous bolus were administered. Because of the high permeability of the rich blood supply, the sublingual route can provide a rapid onset of action.

The intranasal compositions can be administered by any appropriate method according to their form. A composition including microspheres or a powder can be administered using a nasal insufflator device. Examples of these devices are well known to those of skill in the art, and include commercial powder systems such as Fisons Lomudal System. An insufflator produces a finely divided cloud of the dry powder or microspheres. The insufflator is preferably provided with a mechanism to ensure administration of a substantially fixed amount of the composition. The powder or microspheres can be used directly with an insufflator, which is provided with a bottle or container for the powder or microspheres. Alternatively, the powder or microspheres can be filled into a capsule such as a gelatin capsule, or other single dose device adapted for nasal administration. The insufflator preferably has a mechanism to break open the capsule or other device. Further, the composition can provide an initial rapid release of the active ingredient followed by a sustained release of the active ingredient, for example, by providing more than one type of microsphere or powder. Further, alternative methods suitable for administering a composition to the nasal cavity will be well known by the person of ordinary skill in the art. Any suitable method may be used. For a more detailed description of suitable methods reference is made to EP2112923, EP1635783, EP1648406, EP2112923 (the entire contents of which are incorporated by reference herein).

In one embodiment of the present invention, the IL-18 inhibitor or the pharmaceutical composition comprising the IL-18 inhibitor of the invention and as disclosed herein in the various embodiments may be further administered intranasally, i.e. by inhalation and, thus, may be formulated in a form suitable for intranasal administration, i.e. as an aerosol, dry powder formulation or a liquid preparation.

Examples of suitable pharmaceutical carriers, excipients and/or diluents are well known in the art and include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable carriers for liquid formulation are aqueous or non-aqueous solutions, suspensions, dry powder formulations, emulsions or oils. Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, fish-liver oil, another marine oil, or a lipid from milk or eggs.

The present invention also relates to transpulmonary administration by inhalation of the IL-18 inhibitor or the pharmaceutical composition comprising the IL-18 inhibitor of the invention and as disclosed herein in the various embodiments is in dry powder, gaseous or volatile formulations into systemic circulation via the respiratory tract. Absorption is virtually as rapid as the formulation can be delivered into the alveoli of the lungs, since the alveolar and vascular epithelial membranes are quite permeable, blood flow is abundant and there is a very large surface for adsorption. For instance, aerosols may be delivered from pressure-packaged, metered-dose inhalers (MDIs).

The IL-18 inhibitor or the pharmaceutical composition comprising the IL-18 inhibitor of the invention and as disclosed herein in the various embodiments would generally be administered in a mixture with a suitable pharmaceutical excipient, diluent or carrier selected with regard to the chosen means of inhalation and standard pharmaceutical practice.

In another embodiment of the invention, the IL-18 inhibitor formulation, particularly the IL-18BP formulation or the formulation of a pharmaceutical composition comprising the IL18 inhibitor, particularly the IL-18BP, is a dry powder, optionally together with at least one particulate pharmaceutically acceptable carrier, which may be one or more materials known as pharmaceutically acceptable carriers, preferably chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. An especially preferred carrier is lactose, for example lactose monohydrate or anhydrous lactose. The dry powder may be contained as unit doses in capsules of, for example, gelatin or plastic, or in blisters (e.g. of aluminium or plastic), for use in a dry powder inhalation device, which may be a single dose or multiple dose device, preferably in dosage units together with the carrier in amounts to bring the total weight of powder per capsule to from 5 mg to 50 mg. Alternatively, the dry powder may be contained in a reservoir in a multi-dose dry powder inhalation (MDDPI) device adapted to deliver.

Any other therapeutically efficacious route of administration can be used, for example absorption through epithelial or endothelial tissues or by gene therapy wherein a DNA molecule encoding the active agent is administered to the patient (e.g. via an expression vector), which causes the active agent to be expressed and secreted in vivo.

The IL-18 inhibitor or the pharmaceutical composition comprising the IL-18 inhibitor of the invention and as disclosed herein in the various embodiments may be used for treatment of a respiratory disease or disorder as described herein in the various embodiments in human and veterinary medicine for treating humans and animals, including avians, non-human primates, dogs, cats, pigs, goats, sheep, cattle, horses, mice, rats and rabbits.

In a specific embodiment, the present invention provides the IL-18 inhibitor or the pharmaceutical composition comprising the IL-18 inhibitor of the invention as disclosed herein in the various embodiments for use in the treatment of a respiratory disease or disorder as described herein in the various embodiments, wherein the subject is a mammal, in particular the subject is a human.

In another specific embodiment, the pharmaceutical composition of the invention as disclosed herein in the various embodiments is administered in a therapeutically effective amount with a suitable dose of at least a second proinflammatory cytokine inhibitor. In particular said inhibitor is specific for IL-1, IL-6, IL-13, IL-17A, IFNγ or TNFα.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well known conventional methods. Suitable carriers may comprise any material which, when combined with the biologically active compound of the invention, the compound retains the biological activity.

Efforts have been made in the art to chemically modify the barrier properties of skin to permit the penetration of certain agents, enhance the effectiveness of the agent being delivered, enhance delivery times, reduce the dosages delivered, reduce the side effects from various delivery methods, reduce patient reactions, and so forth.

In this regard, penetration enhancers have been used to increase the permeability of the dermal surface to drugs and are often proton accepting solvents such as dimethyl sulfoxide (DMSO) and dimethylacetamide. Other penetration enhancers that have been studied and reported as effective include 2-pyrrolidine, N,N-diethyl-m-toluamide (Deet), 1-dodecal-azacycloheptane-2-one, N,N-dimethylformamide, N-methyl-2-pyrrolidine, calcium thioglycolate, hexanol, fatty acids and esters, pyrrolidone derivatives, derivatives of 1,3-dioxanes and 1,3-dioxolanes, 1-N-dodecyl-2-pyrrolidone-5-carboxylic acid, 2-pentyl-2-oxo-pyrrolidineacetic acid, 2-dodecyl-2-oxo-1-pyrrolidineacetic acid, 1-azacycloheptan-2-one dodecylacetic acid, and aminoalcohol derivatives, including derivatives of 1,3-dioxanes, among others.

Preparations for transmucosal administration may include sterile aqueous or non-aqueous solutions, suspensions, dry powder formulations and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Transmucosal vehicles may include sodium chloride solution, Ringer's dextrose, dextrose and sodium chloride, lactated Ringer's, or fixed oils. Preservatives and other additives may also be present including, for example, antimicrobials, anti-oxidants, chelating agents, and inert gases and the like. In addition, the pharmaceutical composition of the present invention might comprise proteinaceous carriers, like, e.g., serum albumin or immunoglobulin, preferably of human origin.

The IL-18 inhibitor or the pharmaceutical composition comprising the IL-18 inhibitor of the invention as disclosed herein in the various embodiments may be administered topically to body surfaces and, thus, be formulated in a form suitable for topical administration. Suitable topical formulations include gels, ointments, creams, lotions, drops and the like. For topical administration, the pharmaceutical composition of the invention as disclosed herein in the various embodiments is prepared and applied as a solution, suspension, or emulsion in a physiologically acceptable diluent with or without a pharmaceutical carrier.

The IL-18 inhibitor or the pharmaceutical composition comprising the IL-18 inhibitor of the invention and as disclosed herein in the various embodiments may also be administered as controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

Further examples for suitable formulations are provided in WO 2006/085983, the entire contents of which are incorporated by reference herein. For example, the IL-18 inhibitor or the pharmaceutical composition comprising the IL-18 inhibitor of the invention and as disclosed herein in the various embodiments is of the present invention may be provided as liposomal formulations. The technology for forming liposomal suspensions is well known in the art. The lipid layer employed can be of any conventional composition and can either contain cholesterol or can be cholesterol-free. The liposomes can be reduced in size, as through the use of standard sonication and homogenization techniques. Liposomal formulations containing the pharmaceutical composition of the invention as disclosed herein in the various embodiments can be lyophilized to produce a lyophilizate which can be reconstituted with a pharmaceutically acceptable carrier, such as water, to regenerate a liposomal suspension. The pharmaceutical composition of the invention as disclosed herein in the various embodiments can be administered to the subject at a suitable dose. The dosage regimen will be determined by the attending physician and clinical factors. As is well known in the medical arts, dosages for any one subject depend upon many factors, including the subject's size, body surface area, age, the particular compound to be administered, sex, time and route of administration, general health, and other drugs being administered concurrently.

Furthermore, it is envisaged that the IL-18 inhibitor or the pharmaceutical composition comprising the IL-18 inhibitor of the invention might comprise further biologically active agents, depending on the intended use of the pharmaceutical composition. These further biologically active agents may be e.g. antibodies, antibody fragments, hormones, growth factors, enzymes, binding molecules, cytokines, chemokines, nucleic acid molecules and drugs. In a preferred embodiment, the pharmaceutical composition of the present invention may be co-administered with long-acting beta-adrenoceptor agonist (LABA), long-acting muscarinic antagonists (LAMA), steroids, corticosteroid, glucocorticoid and glucocorticoid agonists phosphodiesterase inhibitors, kinase inhibitors, cytokine and chemokine inhibitors, antibiotics, antagonists or protease inhibitors, anti-viral and/or anti-inflammatory drugs or combinations thereof.

The dosage of the IL-18 inhibitor or the pharmaceutical composition comprising the IL-18 inhibitor of the invention as disclosed herein in the various embodiments will depend on the condition being treated, the particular composition used, and other clinical factors such as weight, size and condition of the subject, body surface area, the particular compound or composition to be administered, other drugs being administered concurrently, and the route of administration.

The IL-18 inhibitor or the pharmaceutical composition comprising the IL-18 inhibitor of the invention as disclosed herein in the various embodiments may be administered in combination with other biologically active substances and procedures for the treatment of symptoms associated with IL-18 associated respiratory diseases, particularly virus-induced respiratory diseases. The other biologically active substances may be part of the same composition already comprising the composition according to the invention, in form of a mixture, wherein the composition of the invention and the other biologically active substance are intermixed in or with the same pharmaceutically acceptable solvent and/or carrier or may be provided separately as part of a separate compositions, which may be offered separately or together in form of a kit of parts.

The IL-18 inhibitor or the pharmaceutical composition comprising the IL-18 inhibitor of the invention as disclosed herein in the various embodiments may be administered concomitantly with the other biologically active substance or substances, intermittently or sequentially. For example, the composition according to the invention may be administered simultaneously with a first additional biologically active substance or sequentially after or before administration of said composition. If an application scheme is chosen where more than one additional biologically active substance are administered and at least one composition according to the invention, the compounds or substances may be partially administered simultaneously, partially sequentially in various combinations.

It is thus another object of the present invention to provide for mixtures of the IL-18 inhibitor or the pharmaceutical composition comprising the IL-18 inhibitor of the invention as disclosed herein in the various embodiments, optionally comprising, one or more further biologically active substances in a therapeutically or prophylactically effective amount, as well as to methods of using such a mixture for prevention and/or therapeutic treatment.

The other biologically active substance or compound may exert its biological effect by the same or a similar mechanism as the composition according to the invention or by an unrelated mechanism of action or by a multiplicity of related and/or unrelated mechanisms of action.

Generally, the other biologically active compound may include antibodies raised against and binding to INF-gamma, IL-17A, IL-13, IL-1beta, IL-6, IL-2, IL-4, IL-12, TNF-alpha. In particular, the mixture according to the invention may comprise IL-18BP (IL-18BP) or a pharmaceutical composition comprising IL-18BP (IL-18BP) and a pharmaceutically acceptable carrier and/or excipient according to the invention and as described herein. Suitable dosages of the pharmaceutical composition of the invention as disclosed herein in the various embodiments will vary depending upon the condition, age and species of the subject, and can be readily determined by those skilled in the art. The total daily dosages of the employed in both veterinary and human medicine will suitably be in the range of 0.1 to 10 mg per kilogram.

Further, functional derivatives of the IL-18 inhibitor, particularly the IL-18BP, may be conjugated to polymers in order to improve the properties of the protein, such as the stability, half-life, bioavailability, tolerance by the human body, or immunogenicity. To achieve this goal, IL1-8SP may be linked e.g. to Polyethlyenglycol (PEG). PEGylation may be carried out by known methods, described in WO 92/13095, for example.

Therefore, in another embodiment of the present invention, IL-18BP is PEGylated.

In still another embodiment of the invention, IL-18BP is a fused protein comprising all or part of an IL-18BP, which is fused to all or part of an immunoglobulin, preferably to the constant region (Fc) of an immunoglobulin, and wherein the fused protein is still capable of binding to IL-18. More specifically, the immunoglobulin may be of the IgG1 or IgG2 isotype.

In a further embodiment of the invention, the IL-18BP is PEGylated, fused to all or part of an immunoglobulin, preferably to the constant region (Fc) of an immunoglobulin, and wherein the fused protein is still capable of binding to IL-18. More specifically, the immunoglobulin may be of the IgG1 or IgG2 isotype.

The person skilled in the art will understand that the resulting fusion protein retains the biological activity of IL-18BP, in particular the binding to IL-18. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the IL-18BP sequence and the immunoglobulin sequence. The resulting fusion protein has improved properties, such as an extended residence time in body fluids (half-life), increased specific activity, increased expression level, or the purification of the fusion protein is facilitated.

Preferably, it is fused to heavy chain regions, like the CH2 and CH3 domains of human IgG1, for example. The generation of specific fusion proteins comprising IL-18BP and a portion of an immunoglobulin are described in example 11 of WP99/09063, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero or homomultimeric.

Definitions

The technical terms and expressions used within the scope of this application are generally to be given the meaning commonly applied to them in the pertinent art if not otherwise indicated herein below.

As used in this specification and the appended embodiments, the singular forms “a”, “an”, and “the” include plural referents unless the context clearly dictates otherwise. Thus, for example, reference to “a compound” includes one or more compounds.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effects attributed to the disease. The term “treatment” as used herein covers any treatment of a disease in a subject and includes: (a) preventing a disease, i.e. related to an undesired immune response from occurring in a subject which may be predisposed to the disease; (b) inhibiting the disease, i.e. arresting its development; or (c) relieving the disease, i.e. causing regression of the disease (d) reversing the disease symptoms, i.e. leading to recovery of damaged tissue.

The expression “IL-18 Binding Protein (IL-18BP)” as used herein includes the full-length protein, a mutein, fragment, peptide, functional derivative, functional fragment, fraction, circularly permuted derivative, fused protein comprising IL-18BP, isoform or a salt thereof. The term “free IL-18” as used herein means monomeric, soluble and non-complexed interleukin-18 protein.

The term “functional” and “active” are used herein synonymously and refers to a modified IL-18 inhibitor, particularly a modified IL-18BP, or to a part or fragment of an IL-18 inhibitor, particularly a part or fragment of IL-18BP, or to an equivalent of an IL-18 inhibitor, particularly an equivalent of IL-18BP, which still have/retains the same or essentially the same biological, pharmacological and therapeutic properties as the unmodified or full length IL-18 inhibitor, particularly the unmodified or full length IL-18BP, and can thus be used within the present invention for the treatment of the diseases and disorders as disclosed herein the same way as the unmodified or full length IL-18 inhibitor, particularly as the unmodified or full length IL-18BP. In particular, by “functional” is meant that the modified, partial or equivalent IL-18 inhibitor, particularly the the modified, partial or equivalent IL-18BP, has still retained the IL-18 blocking activity of the unmodified or full length IL-18 inhibitor, particularly the unmodified or full length IL-18BP, in virus-induced infectious processes in the lung and is thus capable of blocking the proinflammatory activity of IL-18 and thus to interrupt the immunopathological cascade responsible for irreversible lung damage.

In various embodiments of the invention, the term “IL-18BP” refers to human IL-18BP, particularly to recombinant human IL-18BP, particularly to isoform a, b, c or d of IL-18BP, particularly isoform a, particularly isoform c, particularly isoform a, b, c or d as shown in SEQ ID NOs 2, and SEQ ID NOs: 3, 4 and 5, but especially the isoform a of IL-18BP as shown in SEQ ID NO: 2 or the isoform c as shown in SEQ ID NO 4.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as [kappa] and [lambda] light chains. Heavy chains are classified as [micro], [Delta], [gamma], [alpha], or [epsilon], and define the antibody's isotype as IgM, IgD, IgG, IgA, and IgE, respectively. Within light and heavy chains, the variable and constant regions are joined by a “J” region of about 12 2 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR.2, FR3, CDR3 and FR4. The assignment of amino acids to each domain is in accordance with the definitions of Kabat Sequences of Proteins of Immunological Interest. The Kabat Complementarity Determining Regions are based on sequence variability and are the most commonly used (National Institutes of Health, Bethesda, Md. (1987 and 1991), or Chothia & Lesk J. Mol. Biol, 196:901-917 (1987)).

Chothia refers instead to the location of the structural loops (Chothia and Lesk J. Mol. Biol. 196:901-917 (1987), Chothia et al. Nature 342:878-883 (1989)).

An alternative system for the assignment of amino acids to each domain is the IMGT system (http://www.imgt.org/IMGTScientificChart/Nomenclature/IMGT-FRCDRdefinition.html). The terms “antibody” or “antibodies” as used herein are art recognized term and are understood to refer to molecules or active fragments of molecules that bind to known antigens, particularly to immunoglobulin molecules and to immunologically active portions of immunoglobulin molecules, i.e. molecules that contain a binding site that immunospecifically binds an antigen. The immunoglobulin according to the invention can be of any type (IgG, IgM, IgD, IgE, IgA and IgY) or class (IgG1, IgG2, IgG3, IgG4, IgA1 and IgA2) or subclasses of immunoglobulin molecule.

The term “Antibody” refers for the purpose of the present invention to an intact immunoglobulin or to an antigen-binding portion thereof that competes with the intact antibody for specific binding. In particular, “Antibodies” are intended within the scope of the present invention to include monoclonal, polyclonal, chimeric, single chain, bispecific or bi-effective, simianized, human and humanized antibodies.

Examples of Antigen-binding portions include, inter alia, Fab, Fab′, F(ab′)2, scFv, dAb and Fv fragments, including the products of an Fab immunoglobulin expression library and epitope-binding fragments of any of the antibodies and fragments mentioned above. Further examples of Antigen-binding portions include complementarity determining region (CDR) fragments, diabodies and polypeptides that contain at least a portion of an immunoglobulin that is sufficient to confer specific antigen binding to the polypeptide.

Such active fragments can be derived from an antibody of the present invention by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. ct al. J. Nucl. Med. 23:1011-1019 (1982); Rousseaux et al. Methods Enzymology, 121:663-69, Academic Press, 1986.

A “patient” or “subject” for the purposes of the present invention is used interchangeably and meant to include both humans and other animals, particularly mammals, and other organisms. Thus, the methods are applicable to both human therapy and veterinary applications. In the preferred embodiment the patient or subject is a mammal, and in the most preferred embodiment the patient or subject is a human.

The expressions “pharmaceutical composition” and “therapeutical composition” are used herein interchangeably in the widest sense. They are meant to refer, for the purposes of the present invention, to a therapeutically effective amount of the active ingredient, i.e. the IL-18BP and, optionally, a pharmaceutically acceptable carrier or diluent.

It embraces compositions that are suitable for the curative treatment, the control, the amelioration, an improvement of the condition or the prevention of a disease or disorder in a human being or a non-human animal. Thus, it embraces pharmaceutical compositions for the use in the area of human or veterinary medicine. Such a “therapeutic composition” is characterized in that it embraces at least one IL-18BP compound or a physiologically acceptable salt thereof, and optionally a carrier or excipient whereby the salt and the carrier and excipient are tolerated by the target organism that is treated therewith.

A “therapeutically effective amount” refers to that amount which provides a therapeutic effect for a given condition and administration regimen. In particular, “therapeutically effective amount” means an amount that is effective to prevent, reverse, alleviate or ameliorate symptoms of the disease or prolong the survival of the subject being treated, which may be a human or non-human animal. Determination of a therapeutically effective amount is within the skill of the person skilled in the art. In particular, in the present case a “therapeutically or prophylactically effective amount” refers to the amount of protein or peptide, mutein, functional derivative, fraction, circularly permuted derivative, fused protein, isoform or a salt thereof, and compound or pharmaceutical composition which, when administered to a human or animal, leads to a therapeutic or prophylactic effect in said human or animal. The effective amount is readily determined by one of skill in the art following routine procedures. The therapeutically effective amount or dosage of a compound according to this invention can vary within wide limits and may be determined in a manner known in the relevant art. The dosage can vary within wide limits and will, of course, have to be adjusted to the individual requirements in each particular case.

The term “transmucosal” administration refers to various administration routes wherein the compound is absorbed by the mucosa of any part of the body. Transmucosal administration comprises, but is not limited to, i.e. intranasal, buccal, oral transmucosal, intratracheal, intraurinary tract, intrarectal, intravaginal, sublingual, intrabronchial, intrapulmonary and transdermal administration.

The definition “pharmaceutically acceptable” is meant to encompass any carrier, excipient, diluent or vehicle, which does not interfere with effectiveness of the biological activity of the active ingredient and that is not toxic to the host to which it is administered.

The term “fused protein” refers to a polypeptide comprising an IL-18BP, or a viral IL-18BP, or a mutein or fragment thereof, fused with another protein, which, e.g., has an extended residence time in body fluids. An IL-18BP or a viral IL-18BP may thus be fused to another protein, polypeptide or the like, e.g., an immunoglobulin or a fragment thereof.

These isoforms, muteins, fused proteins or functional derivatives retain the biological activity of IL-18BP, in particular the binding to IL-18, and preferably have essentially at least an activity similar to IL-18BP. Ideally, such proteins have a biological activity which is even increased in comparison to unmodified IL-18BP. Preferred active fractions have an activity which is better than the activity of IL-18BP, or which have further advantages, like a better stability or a lower toxicity or immunogenicity, or they are easier to produce in large quantities, or easier to purify.

The term “interleukin-18 binding protein” (IL-18BP) comprises also functional equivalents of the IL-18BP, including mutein, functional derivative, fraction, biologically active peptide, circularly permuted derivative, fused protein, isoform and a salt thereof.

In particular, the functional equivalent of the IL-18BP may have a sequence identity of 80%, 85%, 90%, 91%, 92%, 93%, 94%, 95%, 96%, 97%, 98%, 99% to the sequence depicted in SEQ ID NO: 2 and which retains the capability of blocking the proinflammatory activity of IL-18, preferably which retains the IL-18 blocking activity of the IL-18BP in virus-induced infectious processes in the lung and is thus capable of blocking the proinflammatory activity of IL-18 and thus to interrupt the immunopathological cascade responsible for irreversible lung damage.

As used herein the term “muteins” refers to analogs of an IL-18BP, or analogs of a viral IL-18BP, in which one or more of the amino acid residues of a natural IL-18BP or viral IL-18BP are replaced by different amino acid residues, or are deleted, or one or more amino acid residues are added to the natural sequence of an IL-18BP, or a viral IL-18BP, without changing considerably the activity of the resulting products as compared with the wild type IL-18BP or viral IL-18BP. These muteins are prepared by known synthesis and/or by site-directed mutagenesis techniques, high throughput mutagenesis, DNA shuffling, protein evolution techniques, or any other known technique suitable therefore.

Any such mutein preferably has a sequence of amino acids sufficiently duplicative of that of an IL-18BP, or sufficiently duplicative of a viral IL-18BP, such as to have substantially similar activity to IL-18BP. One activity of IL-18BP is its capability of binding IL-18. As long as the mutein has substantial binding activity to IL-18, it can be used in the purification of IL-18, such as by means of affinity chromatography, and thus can be considered to have substantially similar activity to IL-18BP. Thus, it can be determined whether any given mutein has substantially the same activity as IL-18BP by means of routine experimentation comprising subjecting such a mutein, e. g. to a simple sandwich competition assay to determine whether or not it binds to an appropriately labeled IL-18, such as radioimmunoassay or ELISA assay.

Muteins of IL-18BP polypeptides or muteins of viral IL-18BPs, which can be used in accordance with the present invention, or nucleic acid coding therefore, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of IL-18BP polypeptides or proteins or viral IL-18BPs, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g., cysteine residues. Proteins and muteins produced by such deletions and/or insertions come within the purview of the present invention.

“Functional derivatives” as used herein cover derivatives of IL-18BPs or a viral IL-18BP, and their muteins and fused proteins, which may be prepared from the functional groups which occur as side chains on the residues or the N- or C-terminal groups, by means known in the art, and are included in the invention as long as they remain pharmaceutically acceptable, i. e. they do-not destroy the activity of the protein which is substantially similar to the activity of IL-18BP, or viral IL-18BPs, and do not confer toxic properties on compositions containing it.

These derivatives may, for example, include polyethylene glycol side-chains, which may mask antigen sites and extend the residence of an IL-18BP or a viral IL-18BP in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanol or carbocyclic aroyl groups) or O-acyl derivatives of free hydroxyl groups (for example that of seryl or threonyl residues) formed with acyl moieties.

As “functional fragment” of an IL-18BP, or a viral IL-18BP, mutein and fused protein, the present invention covers any fragment or precursors of the polypeptide chain of the IL-18BP protein molecule alone or together with associated molecules or residues linked thereto, e.g., sugar or phosphate residues, or aggregates of the protein molecule or the sugar residues by themselves, provided said fraction has substantially similar activity to IL-18BP.

The term “salts” herein refers to both salts of carboxyl groups and to acid addition salts of amino groups of the IL-18BP molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of IL-18BP, e. g. the ability to bind IL-18.

“Isoforms” of IL-18BP are proteins capable of binding IL-18 or fragment thereof, which may be produced by alternative splicing.

The term “circularly permuted derivatives” as used herein refers to a linear molecule in which the termini have been joined together, either directly or through a linker, to produce a circular molecule, and then the circular molecule is opened at another location to produce a new linear molecule with termini different from the termini in the original molecule. Circular permutations include those molecules whose structure is equivalent to a molecule that has been circularized and then opened. Thus, a circularly permuted molecule may be synthesized de novo as a linear molecule and never go through a circularization and opening step. The preparation of circularly permutated derivatives is described in WO 95/27732.

The expression “abnormal levels of free IL-18” refers to increased or decreased levels of free IL-18 compared to the values detected in body fluids and/or body tissues of a healthy control subject. In particular, these abnormal levels mean increased and detectable values of free IL-18. In particular, said abnormal level of free IL-18 in the body fluids and/or body tissues are levels of free IL-18, which are above the quantification limit, in particular >12 pg/mL and/or above the detection limit, in particular >4 pg/mL of free IL-18, particularly levels of free IL-18, which exceed the level in body fluids and/or body tissues of a healthy control subject by 5%, 10%, 20%, 30%, 40%, 50%, 60%, 70%, 80%, 90%, 100%, or more than 100%. In certain embodiments of the invention the reference or control value is the normal, non-pathologic base value for free IL-18 determined in the patient to be treated. The skilled person is aware that detection and/or quantification limit of free IL-18 may depend on the employed method for determining free IL-18. Therefore, what is possible to be detected may change with the development of improved and/or alternative methods.

The expression “abnormal ratio of free IL-18/IL-18BP” refers to an increased ratio of IL-18 to IL-18BP compared to values found in body fluids and/or body tissues of a healthy control subject. In particular, said abnormal ratio of free IL-18 to IL-18BP in the body fluids and/or body tissues exceeds the ratio in body fluids and/or body tissues of a healthy control subject by 1%, 2.5%, 5%, 10%, 20%, 30%, 40%, 50%, 60%, 70%, 80%, 90%, 100%, or more than 100%. In certain embodiments of the invention the reference or control value is the normal, non-pathologic base value for free IL-18 determined in the patient to be treated.

The expressions “gene silencing” and “post transcriptional gene silencing” mean the suppressive regulation of gene expression by mechanisms others than genetic modification. The silencing occurs by mRNA neutralization on the post transcriptional level, wherein mRNA translation is prevented to form an active gene product, which is in most cases a protein.

The term “predisposition” means the increased susceptibility of a subject for developing a specific disease. In the present case a subject is classified as predisposed if for instance elevated IL-18 level appear in the lung, serum, sputum, broncho-alveolar lavage fluid (BALF) or circulation.

“Alveolar macrophages” are a subtype of macrophages found in the pulmonary alveolus. They often contain granules of exogenous material that they have picked up from the respiratory surfaces. Such black granules are especially common in people, which are long-time exposed to fine dust, fine particles, e.g. like smoker or long-term city dwellers.

A “Th2 cytokine response” mediated by IL-4, IL-5, IL-6, IL-8, IL-10, IL-13, and/or IL-17A, particularly IL-4 and/or IL-8 and/or IL-17A, whereas a “Th1 cytokine response” is mediated by interferon-gamma (IFN-γ), IL-2, and tumor necrosis factor-alpha (TNF-α).

The expression “IL-18/IL-18BP imbalance” relates to the dysregulation of mutual interaction of IL-18 and IL-18BP, which finally leads to an elevated level of unbound IL-18.

A “disease” is a state of health of a subject, particularly a human, wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a symptom of the disease or disorder, the frequency with which such a symptom is experienced by a subject, or both, are reduced.

The terms “dysregulated” or “dysregulation,” as used herein, refer to an impairment in a biological process which in turn may lead to deleterious physiological sequela, or abnormal expression of a gene, nucleic acid, protein, peptide, or other biological molecule. In the case where expression of a gene, nucleic acid, protein, peptide, or other biological molecule is dysregulated, the gene, nucleic acid, protein, peptide, or other biological molecule is expressed, processed, or maintained at levels that are outside what is considered the normal range for that of that gene, nucleic acid, protein, peptide, or other biological molecule as determined by a skilled artisan. Dysregulation of a gene, nucleic acid, protein, peptide, or other biological molecule in a mammal may be determined by measuring the level of a gene, nucleic acid, protein, peptide, or other biological molecule in the mammal and comparing the level measured in that mammal to level measured in a matched population known not to be experiencing dysregulation of that gene, nucleic acid, protein, peptide, or other biological molecule dysregulated. Alternatively, the level may be compared to one measured in the same individual at a different time.

As used herein “endogenous” refers to any material from or produced inside an organism, cell, tissue or system. The term “exogenous” refers to any material introduced from or produced outside an organism, cell, tissue or system.

The terms “inhibit”, “neutralize” or “block” as used herein, have to be understood as synonyms which mean reducing a molecule, a reaction, an interaction, a gene expression, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

### BRIEF DESCRIPTION OF THE SEQUENCES

13-amino acid Linker Sequence of hIL-18BP:
SEQ ID NO: 1:
Glu-Phe-Gly-Ala-Gly-Leu-Val-Leu-Gly-Gly-Gln-
Phe-Met
Amino Acid Sequence of IL-18 Binding Protein
(hIL-18BP),
SEQ ID NO: 2:
isoform a
(SEQ ID NO: 2)
TPVSQTTTAA TASVRSTKDP CPSQPPVFPA AKQCPALEVT
WPEVEVPLNG TLSLSCVACS RFPNFSILYW LGNGSFIEHL
PGRLWEGSTS RERGSTGTQL CKALVLEQLT PALHSTNFSC
VLVDPEQVVQ RHVVLAQLWA GLRATLPPTQ EALPSSHSSP
QQQG
Amino Acid Sequence of IL-18 Binding Protein
(hIL-18BP),
SEQ ID NO: 3:
isoform b
(SEQ ID NO: 3)
TPVSQTTTAA TASVRSTKDP CPSQPPVFPA AKQCPALEVT
WPEVEVPL SWAEGNLAPH PRSPALQPQQ STAAGLRLST
GPAAAQP
Amino Acid Sequence of IL-18 Binding Protein
(hIL-18BP),
SEQ ID NO: 4:
isoform c
(SEQ ID NO: 4)
TPVSQTTTAA TASVRSTKDP CPSQPPVFPA AKQCPALEVT
WPEVEVPLNG TLSLSCVACS RFPNFSILYW LGNGSFIEHL
PGRLWEGSTS RERGSTGTQL CKALVLEQLT PALHSTNFSC
VLVDPEQVVQ RHVVLAQLWV RSPRRGLQEQ EELCFHMWGK
GGLCQSSL
Amino Acid Sequence of IL-18 Binding Protein
(hIL-18BP),
SEQ ID NO: 5:
isoform d
(SEQ ID NO: 5)
TPVSQTTTAA TASVRSTKDP CPSQPPVFPA AKQCPALEVT
WPEVEVPLNG TLSLSCVACS RFPNFSILYW LGNGSFIEHL
PGRLWEGSTS RERGSTGWAE GNLAPHPRSP ALQPQQSTAA
GRLSTGPAAA QP

### BRIEF DESCRIPTION OF THE FIGURE

FIG. 1: Correlation plots. Scatterplots of all the pairs of variables, accompanied by a loess curve indicating the trend. The correlations between the variables are indicated in the top-right triangle (font size is proportional to the absolute value of the correlation)

### EXAMPLES

### Example 1

### Free IL-18, Total IL-18 and IL-18BP Levels in SARS-Cov-2 Patients

1.1 Study Population

37 serum samples obtained from hospitalized patients with confirmed SARS-CoV-2 infection by reverse-transcriptase-polymerase chain reaction (RT-PCR) undergoing systemic inflammatory reaction and in need of respiratory assistance were analyzed for C-reactive protein (CRP), ferritin, glutamic oxaloacetic transaminase (GOT, also called AST), total IL-18, free IL-18 and IL-18BP levels.

In the clinical course of SARS-CoV-2 infection some patients deteriorate and progress to a status of uncontrolled inflammatory reaction that has been called “cytokine release syndrome”. It has been acknowledged that the body's detrimental inflammatory response is the main factor that leads to multiple organ failure and poor prognosis. In the clinical setting the increase of inflammatory markers such as CRP and ferritin are the main tools to assess the increasing inflammation.

1.2. Assay Design

1.2.1 Total IL-18:

Total IL-18 in the serum samples was determined with the Human IL-18 ELISA kit (Medical & Biological Laboratories Co., LTD. Code No. 7620). This kit is based on the quantitative sandwich enzyme immunoassay technique. The assay referenced above uses two monoclonal antibodies against two different epitopes of human IL-18. In wells coated with a first anti-human IL-18 monoclonal antibody, samples or reference standards are incubated. After a washing step, a second anti-human IL-18 monoclonal antibody which is conjugated to horseradish peroxidase is added into the microwell and incubated. After another washing step, the peroxidase substrate is mixed with the chromogen and allowed to incubate for an additional period of time. An acid solution is then added to each well to terminate the enzyme reaction and to stabilize the developed colour. The optical density (O.D.) of each well is measured at 450 nm using a microplate reader. The concentration of human IL-18 is calibrated from a dose response curve based on reference standards.

1.2.2 Free IL-18:

Free IL-18 in the serum samples was determined with the assay as described in WO 2015/032932 and WO 2016/139297. For this analysis the limit of quantification was 12 pg/mL and the limit of detection was 4 pg/mL.

Microplate wells are coated with an appropriate volume phosphate buffer saline solution containing recombinant human IL-18BP. Plates are incubated for a period of time at 4° C. and then stabilized with a blocking buffer containing bovine serum albumin or other appropriate blocking agents. Once the reaction is finished, microplates are sealed and stored at 4° C. until used for detection of free IL-18. Microplates can also be dried in a stabilizing solution allowing storage at room temperature and then be reconstituted by hydration when needed for assay.

For a final reaction volume of 100 μl, first 80 μl of biotin/antibody conjugate are dispensed. Samples or biological fluids containing free IL-18 are tested with the IL-18BP coated microplates. After that, 20 μl sample volume containing biological fluid or standard is dispensed per microplate well. The free IL-18 standard concentrations range between 4.2 pg/ml to 3000 pg/ml. Standard concentrations were prepared from commercially available recombinant human IL-18. The plates are sealed and then incubated under gentle shaking for free IL-18 capture. A suitable period of time is allowed for the reaction ranging from minutes to hours at room temperature, 37° C. or other temperatures that do not affect the stability of the samples and reagents. The microplate wells are washed extensively with the appropriate buffer and then, 100 μl buffer developing mixture is added to each well. The developing mixture contains a streptavidin-conjugated enzyme such as peroxidase or alkaline phosphatase. The microplate wells are sealed and the reaction is allowed for a suitable period of time ranging from minutes to hours at room temperature, 37° C. or other temperatures that do not affect the stability of the samples and reagents. The resulting reactions are then monitored with a microplate reader at an appropriate nanometer wavelength for absorbance or fluorescence of the produced reagent.

1.2.3 Ferritin:

Ferritin in the serum samples was determined with the ECLIA Test Kit from Roche Diagnostics, analyzed on Cobas 6000 e601 from Roche Diagnostics

The test method is based on the sandwich-principle. As a first step of the reaction, the analyte (ferritin) forms a sandwich-complex with on the one hand a monoclonal ferritin-specific antibody, labelled with biotin and on the other hand a monoclonal ferritin-specific antibody, labelled with a ruthenium-complex. After addition of streptavidin-coated magnetic microparticles to the solution, the sandwich-complex binds via biotin-streptavidin interaction to the solid phase. By magnetic separation the microparticles are fixed to the surface of an electrode. Application of voltage induces chemiluminescent emission which is measured by a photomultiplier. The concentration of ferritin in the samples is then extrapolated from a standard curve.

1.2.4 CRP:

CRP in the serum samples was determined with an immunoturbidimetric test from Roche Diagnostics, analyzed on Cobas 6000 c501 from Roche Diagnostics

The human CRP test kit is based on an immunoturbidimetric method. CRP in the samples agglutinates with latex particles coated with monoclonal anti-CRP antibodies. The aggregates are determined turbidimetrically.

1.2.5 AST/GOT:

AST/GOT (glutamic oxaloacetic transaminase, also named Aspartate transaminase or AST), in the serum samples was determined with an enzymatic test from Roche Diagnostics, analyzed on Cobas 6000 c501 from Roche Diagnostics.

AST catalyzes the reaction between L-alanine and 2-oxoglutarate and the produced pyruvate is reduced by Lactate Dehydrogenase in presence of NADH to L-lactate and NAD+.

1.2.6 Biostatistical Analysis:

All statistical analyses were performed using the R statistical package (version 4.0.3). The correlation coefficient indicates the (linear) correlations between all pairs of variables. Correlation coefficients are between −1 and 1; a correlation coefficient around 0 indicates no linear relationship between two variables (a cloud of points with no obvious structure linking the two variables); the closer the value approaches 1, the stronger the positive linear association. Similarly, the closer the value approaches −1, the stronger the negative linear association (when one value increases, the other one tends to decrease).

1.3 Results

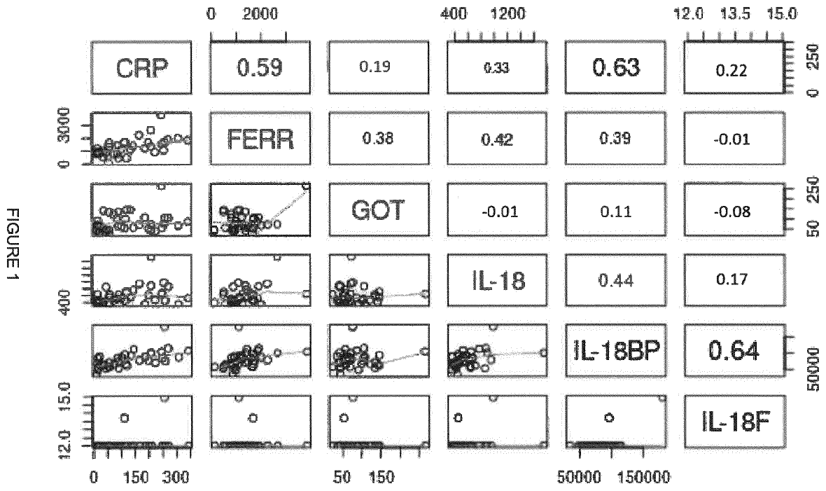

The results of the above described assays are summarized in Table 1 and FIG. 1.

1.4 Discussion

All values for total IL-18 were above values of healthy controls (<260 pg/mL) ranging from 341 to 1734 pg/mL with an average of 585.5 pg/mL and a median of 520 ng/mL. In addition, three patients showed levels of free IL-18 above the quantification limit and eighteen patients were reported with values of free IL-18 above the limit of detection (between 4 pg/mL and 12 pg/mL). Healthy patients had undetectable free IL-18 amounts. All values for IL-18BP were above the values of healthy controls (2000 and 3000 pg/mL) ranging from 34506 to 180288 pg/mL with an average of 80254.6 pg/mL and a median of 77532 pg/mL.

Uncontrolled systemic inflammatory reactions were shown by high levels of ferritin ranging from 106 to 3825 ng/mL with an average of 1306 ng/mL and a median of 1178 ng/mL (values of healthy controls 30-400 ng/mL), high levels of CRP ranging from 8 to 341 μg/mL with an average of 127 ng/mL and a median of 106 ng/mL (values of healthy controls <5 μg/mL) and high levels of GOT ranging from 25 to 266 U/L with an average of 83 U/L and a median of 71 U/L (values of healthy controls 5-40 U/L). These uncontrolled systemic inflammatory reactions result from the activation of macrophages and lymphocytes and are driven, at least partially, by high levels of IL-18 and the appearance of free IL-18. Macrophage activation syndrome is a life-threatening complication of the systemic inflammatory disorders with unremitting fever, hyperferritinemia, high CRP, pancytopenia, coagulopathy and liver damage as shown by the high levels of serum enzymes released from damaged hepatocytes (AST/GOT, normal ranges age and gender dependent).

Since the serum concentrations of these analytes were greater than the concentrations observed in healthy individuals (normal levels), cut off values for each parameter were defined so that the set of values was divided in two groups (below and above threshold) of similar sizes. The below and above threshold groups correspond to medium and high levels, respectively.

Among the 22 patients presenting high (hi) levels of Ferritin (>1000 μg/L; FERRhi):
20 (91%) also presented high levels of CRP (>90 mg/L; CRPhi)
16 (73%) also presented high levels of GOT (>60 U/L; GOThi)
16 (73%) also presented high levels of total IL-18 (>500 pg/mL; IL-18hi)
19 (86%) also presented high levels of IL-18BP (>72000 pg/mL; IL-18BPhi)

Among the 14 patients presenting medium (med) levels of Ferritin (<1000 μg/L; FERRmed):
Only 3 (21%) presented high levels of CRP (CRPhi)
6 (43%) presented high levels of GOT (GOThi)
Only 3 (21%) presented high levels of total IL-18 (IL-18hi)
Only 2 (14%) presented high levels of IL-18BP (IL-18BPhi)

In 27 out of 37 patients, IL-18 and IL-18BP levels co-segregated:
Only 4 patients showed high IL-18 and medium IL-18BP
Only 6 patients showed high IL-18BP and medium IL-18

The systemic inflammation that these Covid-19 patients experienced was reflected by above the normal levels of serum Ferritin, CRP and GOT. When these patients were distributed into high or medium levels subgroups, FERRhi patients co-segregate with CRPhi patients, and to a lesser extent to GOThI patients.

This inflammation was accompanied by the induction of IL-18 pathway, as all inflamed patients presented above the normal levels of total IL-18 and IL-18BP. Moreover, the patients exhibiting the highest inflammatory condition (FERRhi) were the ones having high total IL-18 and IL-18BP levels. The total IL-18 co-segregates with the inflammatory status as FERRhi IL-18hi patients represented a major fraction of FERRhi patients (16 out of 22), and also a major fraction of all patients presenting high IL-18 levels (16 out of 19). The same was found for IL-18BP, with FERRhi IL-18BPhi representing 19 out of 22 FERRhi and 19 out of 21 IL-18BPhi. Therefore, the inflammation observed in these severe Covid-19 patients is associated with above the normal levels of both total IL-18 and IL-18BP, and its extent corresponds to the highest levels in these parameters.

The IL-18 induction leads to an increased expression in both IL-18 and IL-18BP, and the data revealed that these parameters largely co-segregate: only 4 out of 37 patients exhibited IL-18hi and IL-18BPmed and 6 patients had IL-18med and IL-18BPhi. Nevertheless, free IL-18 is detected in the majority (24 out of 37) of serum samples. This suggests that the observed increased amounts of IL-18BP relative to the ones of IL-18 are not sufficient and that a large molar excess of IL-18BP over IL-18 is needed to displace the complex equilibrium and keep IL-18 in a complexed inactive status, thus eliminating free IL-18 species.

An additional biostatistical analysis of the results presented in Table 1 was performed in order to determine whether there exists a correlation between these measured parameters, in order to define sub-groups of patients sharing similar characteristics.

The correlation plots indicated that some of the highest correlations are found between IL-18BP and CRP (0.63), as well as between FERR and CRP (0.59) and between IL-18BP and FERR (0.39). The scatterplots showing these pairs of variables indicate indeed a clear relationship between these variables (see FIG. 1). Other high correlations are observed between IL-18BP and free IL-18 (IL-18F) (0.64), between IL-18BP and IL-18 (0.44) and between FERR and IL-18 (0.42). However, their corresponding correlation plots tend to indicate that these higher correlation values are mostly driven by a few outlier points. The Pearson correlation is known to be sensitive to such outliers.

Detection of above the normal serum levels in the inflammatory biomarkers FERR, CRP and GOT confirmed that the severe Covid-19 patients tested in this study experienced a systemic inflammation. Moreover, patients presenting high FERR levels (FERRhi) also had high CRP levels, while they co-segregated to a lesser extent with patients presenting high GOT levels. This was confirmed by the biostatistical analysis, since one of the highest correlations was found between FERR and CRP (0.59), whereas no good correlation was observed between FERR and GOT (0.38, but driven by only one outlier point), nor between CRP and GOT (0.19). Thus, within the present invention, an increased FERR level can be used as additional marker either alone or together with CRP as a marker.

This study revealed that severe Covid-19 patients exhibit not only high serum concentrations in IL-18, but also high levels of IL-18BP. The association between hyperinflammation and the induction of the IL-18 pathway is reflected by the fact that the most inflamed patients (FERRhi) presented high levels of IL-18 and IL-18BP. This is further supported by the statistical analysis which revealed high correlations between CRP and IL-18BP (0.63); FERR and IL-18BP (0.39), as well as between FERR and IL-18 (0.42).

Therefore, the hyperinflammation is associated with the induction of the IL-18 pathway, with both IL-18 and IL-18BP levels being increased. Still, the patients' population is heterologous and for a small fraction of them, IL-18 and IL-18BP do not co-segregate. This is reflected by a correlation between both parameter (0.44) that is only driven by a few outlier points. Moreover, even though the production of both molecules is increased, the amounts of IL-18BP is not sufficient to bind and neutralize IL-18, as seen by the appearance of free IL-18 species. It could be that this is due to a dynamic equilibrium between bound and free IL-18 and a large molar excess of IL-18BP over IL-18 is needed to stabilize the complex, or to the generation of non-functional IL-18BP species in some patients. In both cases, free IL-18 species can be detected and the pathogenically appearance of free IL-18 in the serum of these patients reveals that the inflammation they are experiencing is largely uncontrolled.

In conclusion, high levels of total IL-18 and IL-18BP were found in SARS-Cov-2 patients undergoing systemic inflammatory reaction and in need of respiratory assistance. In addition, two patients exhibited levels of free IL-18 above the quantification limit (>12 pg/mL) and eighteen patients presented detectable levels (>4 pg/mL), while still below the quantification limit. The results indicate a strong implication of the IL-18 pathway in patients with severe SARS-Cov-2 infection and support the use of IL-18 inhibitor such as rhlL-18BP (Tadekinig alfa) to treat such patients. Tadekinig also has proven effect in patients with diseases related to the IL-18 pathway, the same mechanism that is of high relevance for future treatments of patients with severe SARS-Cov-2 infection because the elevated IL-18 levels in such patients are linked with a cytokine release syndrome with the high probability of a fatal outcome.

### Example 2

### rhIL-18BP Compassionate Use Treatment for Hospitalized Patient with Severe S S-CoV-2 Infection

For a group of selected patients, IL-18 blockade with rhlL-18BP (Tadekinig alfa) is proposed. SARS-CoV-2 patients at various stages of the disease may benefit from the treatment. In particular, however not limited thereto, patients undergoing severe ARDS with presence or not of organ failures and a biological reaction characterized by very high levels of ferritin, cytopenias and coagulopathies, mimicking MAS may benefit more of this targeted treatment.

2.1. Objectives

Primary Objective:

To show the clinical and biological efficacy of IL-18 blockade with Tadekinig alfa in SARS-CoV-2 patients with underlying MAS like reaction.

Secondary Objective:

To show the safe use of IL-18 blockade with Tadekinig alfa in SARS-CoV-2 patients with severe disease.

2.2. Stud Design

Compassionate use cases studies including around 5 patients.

2.3. Study Population

2.3.1 Inclusion Criteria
Males or females≥18 years of age.
Hospitalized patients with confirmed SARS-CoV-2 diagnosis by PCR.
Patients with acute respiratory failure (including patients in need of mechanical ventilation).
Patients with worsening clinical or radiologic/CT condition despite standard of care or antiviral agents, or
Patients with new onset organ failures as shown by >3 points on the SOFA score, or
Patient with increased need of vasopressors, and/or
Patients with increasing levels of systemic biomarkers: Ferritin, thrombocytopenia, liver enzymes, low fibrinogen, IL-6, d-dimers.

2.3.2 Exclusion Criteria
Physician decision that enrolment is not in the patient's best interest.

2.4 Study Procedures

2.4.1 Duration

One to eight weeks treatment will be followed by a 4-week follow-up period for safety assessments. Data management, statistical and study report will take approximately 4 more months.

2.4.2 Study Drug

IL-18BP, in particular rhlL-18BP (Tadekinig alfa) isoform a as shown in SEQ ID NO: 2.

2.4.3 Composition

The drug product formulation (recombinant human interleukin 18 Binding protein (rhlL-18BP as shown in SEQ ID NO: 2) will have a strength of 80 mg, and is prepared in a sterilized solution for injection containing sodium chloride, 7 mg per vial, sodium dihydrSEQogen phosphate monohydrate approx. 1.0 mg per vial, disodium phosphate dehydrate approx. 2.4 mg per vial, sodium hydroxide and O-phosphoric acid 85% to adjust to pH 7.0, water for injection up to 1 ml.

Glass vials containing 1 ml of the injection volume and 80 mg of the recombinant molecule (rhIL-18BP) will be the administration unit.

2.4.4 Dose/Route Regime

Open label administration of Tadekinig alfa (rhlL-18BP), at 2 mg/Kg body weight (BW) every other day for three weeks added on to the standard of care.
Permitted co-medications and standard medical care:
   O2 supplementation, non-invasive or invasive ventilation, antibiotics, vasopressor support, antiviral agents, anti-inflammatory and immunosupressant.

The product comes as a solution to be administered by the s.c. route. Glass vials contain 1 ml with a 15% overfill with a dose of 80 mg of the active product. Patients in the 160 mg and 320 mg cohorts will receive separate s.c. injections of 1 ml each: 2 injections of 1 mL for the 160 mg dose and 4 injections of 1 mL for the 320 mg dose. A separate syringe must be used for each injection.

The site of the s.c. injection should be alternated e.g. the outside of the thighs and the various quadrants of the anterior abdominal wall. The separate injections that constitute a single dosage of study drug should be administered within the same body region but not at the exact same injection site.

2.4.5 Clinical and Biological Monitoring During Follow-Up
Daily patient assessment by trained nurses included:
Presence/absence of fever, respiratory rate, systolic blood pressure, level of blood oxygenation, lung and cardiac auscultation.
Daily laboratory controls include:
   White cell counts, platelet counts, CRP, ferritin), serum creatinine, ALT, AST, LDH, Creatinine kinase.
Required co-medications.

2.5. Outcome Measures.

2.5.1 Clinical Assessment

The seven-category ordinal scale will be utilized for clinical assessment, and two-point improvement or live discharge from hospital, will be considered a positive response to treatment.

The seven-category ordinal scale consisted of the following categories:
1, not hospitalized with resumption of normal activities;
2, not hospitalized, but unable to resume normal activities;
3, hospitalized, not requiring supplemental oxygen;
4, hospitalized, requiring supplemental oxygen;
5, hospitalized, requiring nasal high-flow oxygen therapy, noninvasive mechanical ventilation,
Or both;
6, hospitalized, requiring ECMO, invasive mechanical ventilation, or both; and
7, death.

2.5.2 Biological Assessment
Ferritin
CRP
Thrombocytes
Liver enzymes (ALT, AST)
LDH
Creatinine 2.6 Safety Evaluation.

Safety outcomes will include adverse events that occur during treatment, serious adverse events, and premature discontinuation of treatment.

TABLE 1
Table 1: Analysis of serum samples obtained from hospitalized
patients with confirmed SARS-Cov-2 infection

| | | CRP | Ferritin | GOT (AST) | Total IL-18 | IL-18BP | Free IL-18 | Free IL18 |
|---|---|---|---|---|---|---|---|---|
| Sample No | age | μg/mL | ng/mL | U/L | pg/mL | pg/mL | pg/mL | Raw data |
| 0880237562 | 31 | 57.3 | 1637 | 25 | 629 | 58292 | BLQ | 5.06 |
| 0880237563 | 56 | 222.5 | 937.8 | 40 | 467 | 71575 | BLQ | 8.82 |
| 0880237564 | 55 | 134.4 | 1116 | 146 | 553 | 93121 | BLQ | 9.12 |
| 0880237565 | 58 | 84.7 | 780.4 | 105 | 496 | 85314 | BLQ | 6.46 |
| 0880237566 | 57 | 32.9 | 555.8 | 56 | 358 | 66712 | BLD | 2.42 |
| 0880237567 | 53 | 119.3 | 480.9 | 146 | 704 | 61585 | NVR | 13.67 |
| 0880237568 | 54 | 54.8 | 105.6 | 44 | 392 | 58773 | BLD | 4.75 |
| 0880237569 | 55 | 90.5 | 459 | 135 | 627 | 54668 | BLQ | 7.40 |
| 0880237570 | 59 | 341.7 | 1833 | 87 | 520 | 107536 | BLQ | 6.93 |
| 0880237571 | 60 | 307.1 | 1998.5 | 67 | 341 | 87701 | NVR | 12.41 |
| 0880237572 | 60 | 8.2 | 841.6 | 44 | 434 | 34506 | BLD | 3.97 |
| 0880237573 | 58 | NVR | NVR | NVR | 412 | 51796 | NVR | 0.41 |
| 0880237574 | 51 | 207.9 | 1372 | 42 | 882 | 100828 | BLQ | 9.27 |
| 0880237575 | 59 | 165.6 | 2244.5 | 72 | 941 | 78975 | 12 | 11.93 |
| 0880237576 | 60 | 88.5 | 1463 | 64 | 520 | 74480 | BLQ | 6.00 |
| 0880237577 | 55 | 35.7 | 1035 | 142 | 412 | 64502 | BLD | 3.20 |
| 0880237578 | 51 | 100.8 | 1134 | 62 | 521 | 87058 | BLQ | 6.78 |
| 0880237579 | 45 | 55.8 | 1358 | 135 | 561 | 72331 | BLQ | 5.37 |
| 0880237580 | 48 | 14.3 | 940.2 | 70 | 425 | 5687.2 | BLD | 3.35 |
| 0880237581 | 35 | 269.8 | 1890 | 106 | 861 | 114562 | BLQ | 5.68 |
| 0880237582 | 49 | 12.5 | 812 | 43 | 378 | 70600 | BLD | 3.04 |
| 0880237583 | 50 | 14.8 | 948.1 | 92 | 349 | 48335 | BLD | 2.88 |
| 0880237584 | 55 | 258.6 | 1695.5 | 104 | 451 | 76681 | BLD | 2.89 |
| 0880237585 | 51 | 20.9 | 834.6 | 59 | 384 | 63778 | BLD | 1.96 |
| 0880237586 | 64 | 245.4 | 3825 | 266 | 639 | 104232 | BLQ | 6.78 |
| 0880237587 | 67 | 13.2 | 1246 | 72 | 644 | 84531 | BLD | 1.49 |
| 0880237588 | 60 | 208.4 | 2642 | 73 | 1734 | 99963 | BLQ | 6.78 |
| 0880237589 | 69 | 43.5 | 862.6 | 44 | 847 | 53424 | BLQ | 6.93 |
| 0880237597 | 45 | 20.4 | 901 | 36 | 457 | 89932 | BLD | 1.49 |
| 0880237598 | 55 | 114.6 | 1680 | 100 | 455 | 68040 | BLQ | 6.62 |
| 0880237599 | 58 | 248.3 | 1575 | 56 | 666 | 85146 | BLQ | 6.15 |
| 0880237600 | 55 | 197.9 | 1678 | 53 | 615 | 78383 | BLD | 2.26 |
| 0880237601 | 53 | 143.6 | 1406 | 57 | 762 | 112127 | BLQ | 4.52 |
| 0880237602 | 57 | 72.3 | 769.5 | 123 | 455 | 66345 | BLD | 2.20 |
| 0880237603 | 60 | 254.8 | 1080 | 77 | 974 | 180288 | 15 | 14.93 |
| 0880237604 | 74 | 111.5 | 1662 | 54 | 445 | 96347 | 14 | 13.69 |
| 0880237605 | 76 | 186 | 1222 | 108 | 352 | 88120 | BLQ | 11.25 |

NVR: non valid result
BLD: Below limit of detection, <4 pg/mL
BLQ: Below limit of quantification, between 4 pg/mL and 12 pg/mL.

1. A method of treating a respiratory disease in a subject, which disease is caused by virus-induced infection, the method comprising steps of administering to the subject an IL-18 inhibitor.

2. The method of claim 1, wherein the respiratory disease is Acute Respiratory Distress Syndrome (ARDS).

3. The method of claim 1, wherein the virus infection is caused by a virus selected from the group consisting of Rhinovirus, RSV, Influenza virus, Parainfluenza virus, Metapneumovirus, Coronavirus, Enterovirus, Adenovirus, Bocavirus, Polyomavirus, Herpes simplex virus, and Cytomegalovirus.

4. The method of claim 3, wherein the Coronavirus is of the genus α-CoV, β-CoV, γ-CoV or δ-CoV.

5. The method of claim 3, wherein the Coronavirus is selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-CoV or “novel coronavirus 2012”), Severe acute respiratory syndrome coronavirus (SARS-CoV or “SARS-classic”), and Severe acute respiratory syndrome coronavirus 2 (SARS-CoV-2 or “novel coronavirus 2019”).

6. The method of claim 3, wherein the Influenza virus is of type A, B, C or D, in particular of type A.

7. The method of claim 3, wherein the Coronavirus-induced disease is selected from the group consisting of Middle East Respiratory Syndrome (MERS), Severe Acute Respiratory Syndrome (SARS) and COVID-19.

8. The method of claim 3, wherein the Coronavirus is Severe acute respiratory syndrome coronavirus 2 (SARS-CoV-2 or “novel coronavirus 2019”) or a variant thereof and the Coronavirus-induced disease is COVID-19.

9. The method of claim 1, wherein treatment is achieved and/or supported by blocking the proinflammatory activity of IL-18.

10. The method of claim 1, wherein the IL-18 inhibitor is:
(a) an IL-18 binding protein (IL-18BP);
(b) a human IL-18BP (hIL-18BP); or
(c) a recombinant human IL-18BP (rhIL-18BP),
including any functional equivalent or functional part thereof which retains the capability of blocking the proinflammatory activity of IL-18.

11.-12. (canceled)

13. The method of claim 10, wherein said human IL-18BP is selected from isoform a, b, c and d of human IL-18BP, particularly isoform a as in SEQ ID NO: 2, isoform b as in SEQ ID NO: 3, isoform c as in SEQ ID NO: 4 or isoform d as in SEQ ID NO: 5, including any functional equivalent or functional part of isoforms a, b, c and/or d which retains the capability of blocking the proinflammatory activity of IL-18.

14. The method of claim 10, which is an IL-18BP as shown in SEQ ID NO: 2, including any functional equivalent or functional part thereof which retains the capability of blocking the proinflammatory activity of IL-18.

15. The method of claim 10 wherein
(a) the functional equivalent has a sequence identity of 80%, 85%, 90%, 91%, 92%, 93%, 94%, 95%, 96%, 97%, 98%, 99% to the sequence depicted in SEQ ID NO: 2 and retains the capability of blocking the proinflammatory activity of IL-18; or
(b) the functional equivalent or functional part thereof includes a mutein of IL-18BP, a fragment, a peptide, a functional derivative, a functional fragment, a fraction, a circularly permuted derivative, a fused protein comprising IL-18BP, an isoform or a salt thereof which retains the capability of blocking the proinflammatory activity of IL-18.

16. (canceled)

17. The method of claim 10, wherein the IL-18 inhibitor comprises in addition to the IL-18 binding protein (IL-18BP), N-terminal and/or C-terminal deletion variants of IL-18BP in an amount of up to 0.01%, 0.05%, 0.1%, 0.25%, 0.5%, 1%, 2.5%, 5%, 7.5%, 10%, 15%, 20%, 30%, or 40%.

18. The method claim 17, wherein said deletion variants comprise deletions of between 1 and 5 amino acid residues at the C-terminal end of the IL-18BP and/or between 1 and 30 amino acid residues at the N-terminal end of the IL-18BP.

19. (canceled)

20. The method of claim 1, wherein the body fluids and/or body tissues of the subject to be treated have been quantified to have abnormal levels of free IL-18, which exceed the level of free IL-18 in body fluids and/or body tissues of a healthy control subject by 5%, 10%, 20%, 30%, 40%, 50%, 60%, 70%, 80%, 90%, 100%, or more than 100%, using an assay capable of detecting free IL-18 in body fluids and/or body tissues, said assay comprising IL-18BP or an antibody or a functional part thereof, which antibody or active part thereof binds to IL-18 at the binding site of IL-18BP or in the vicinity of the binding site of IL-18BP, but does not bind IL-18/IL-18BP complexes.

21. The method of claim 20, wherein
(a) the level of free IL-18 is above the quantification limit of >12 pg/mL, and/or above the detection limit of >4 pg/mL; or
(b) the level of free IL-18 is two to three times above the level of a healthy control subject.

22. (canceled)

23. The method of claim 1, wherein the subject has a level of ferritin above 400 ng/mL, preferably above 1000 ng/mL.

24. The method of claim 20, wherein the assay for quantifying the level of free IL-18 in the body fluids and/or body tissues comprises the following steps:
a) bringing a sample of body fluid and/or body tissue suspected to contain free IL-18 into contact with the IL-18 inhibitor as defined in any one of claims 9 to 19 as the capturing molecule for free IL-18;
b) allowing the IL-18 inhibitor to bind free IL-18;
c) detecting the binding of the IL-18 inhibitor and determining the amount of free IL-18 in the sample.

25. The method of claim 20, wherein the body fluids and/or body tissues are selected from the group consisting of broncho-alveolar lavage fluid (BALF) circulation fluids, secretion fluids, biopsy, and homogenized tissue, particularly serum, urine, tear, saliva, bile, sweat, exhalation or expiration, sputum, bronchoalveolar fluid, sebum, cellular, gland, mucosa or tissue secretion.

26. (canceled)

27. The method of claim 1, wherein said IL-18 inhibitor or composition is administered to a subject in need thereof, wherein the administration is
(a) a single dose/day, in multiple doses/day, in multiple doses/week or in multiple doses/month;
(b) one dose per week, in two doses per week, three doses per week, four doses per week, five doses per week, six doses per week, or seven doses per week;
(c) every 24 hours to 48 hours; or
(d) a single dose every other day, for example, over three weeks.

28.-30. (canceled)

31. The method of claim 1, wherein a single dose comprises between 0.5 mg of IL-18 inhibitor/kg body weight and 10 mg IL-18 inhibitor/kg body weight.

32. The method of claim 1, wherein a single dose of between 0.5 mg IL-18 inhibitor/kg body weight and 5 mg IL-18 inhibitor/kg body weight is administered every 24 or 48 h.

33. The method of claim 10, wherein the IL-18 inhibitor is a recombinant human IL-18BP (rhIL-18 BP), including any functional equivalent or functional part thereof, which retains the capability of blocking the proinflammatory activity of free IL-18, wherein the recombinant human IL-18BP (rhIL-18 BP) is administered in a single dose every other day of 2 mg/kg body weight.

34. The method of claim 1, wherein the subject, is a human with confirmed SARS-CoV-2 infection undergoing systemic inflammatory reaction and in need of respiratory assistance.

35. The IL-18 inhibitor claim 1, wherein the subject suffers from SARS-Cov-2 showing uncontrolled systemic inflammatory reactions and/or high levels of total IL-18 and/or total IL-18BP and/or detectable levels of free IL-18.

36. The method of claim 1, wherein the IL-18 inhibitor and/or the composition are used in co-medication with an anti-viral agent an anti-inflammatory agent, an immunosuppressant, a monoclonal antibody a cocktail of monoclonal antibodies, a vasopressor, an anticoagulant and/or, a vasodilator, and/or a mucolytic.

37. The method of claim 36, wherein the anti-viral agent is selected from the group consisting of Remdesivir, Chloroquine, Hydroxychloroquine, Lopinavir, Ritonavir, interferon-β, Interferon-α, and Ivermectin.

38. The method of claim 36, wherein the anti-inflammatory agent and the immunosuppressant is selected from the group consisting of Corticosteroids, intravenous immunoglobulin, Tocilizumab (anti-IL-6 receptor), sarilumab (anti-IL-6 receptor), canakinumab (anti-IL-1 beta) anakinra (recombinant IL-1ra), Emapalumab (anti-IFNγ), and JAK inhibitors.

39. The method of claim 36, wherein the monoclonal antibody is selected from REGN-COV2, LY-COV55, and CT-P59.

40. The method of claim 1, wherein the subject to be treated is a mammal or a human.

41.-47. (canceled)


SEQUENCE LISTING (6 sequences)

SEQ ID NO: 1 - protein, 13 aa, Artificial Sequence
  13-amino acid Linker Sequence of hIL-18BP
EFGAGLVLGGQFM

SEQ ID NO: 2 - protein, 164 aa, Artificial Sequence
  Amino Acid Sequence of IL-18 Binding Protein (hIL-18BP)
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 3 - protein, 85 aa, Artificial Sequence
  Amino Acid Sequence of IL-18 Binding Protein (hIL-18BP)
TPVSQTTTAATASVRSTKDPCPSQPPVFPAAKQCPALEVTWPEVEVPLSWAEGNLAPHPRSPALQPQQSTAAGLRLSTGPAAAQP

SEQ ID NO: 4 - protein, 168 aa, Artificial Sequence
  Amino Acid Sequence of IL-18 Binding Protein (hIL-18BP)
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGKGGLCQSSL

SEQ ID NO: 5 - protein, 132 aa, Artificial Sequence
  Amino Acid Sequence of IL-18 Binding Protein (hIL-18BP)
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGWAEGNLAPHPRSPALQPQQSTAAGRLSTGPAAAQP

SEQ ID NO: 6 - protein, 36 aa, Artificial Sequence
  Synthesized Peptide Construct
GLPHEGLYALAGLYLEVALLEGLYGLYGLNPHEMET